(19) United States
(12) Patent Application Publication
(10) Pub. No.: US 2026/0085122 A1
(43) Pub. Date: Mar. 26, 2026

(54) ANTI-B7H3 ANTIBODIES AND METHODS OF USE

(71) Applicant: BeOne Medicines I GmbH, Basel (CH)

(72) Inventors: Liang QU, Beijing (CN); Yao DING, Beijing (CN); Ya ZHANG, Beijing (CN)

(73) Assignee: BeOne Medicines I GmbH, Basel (CH)

(21) Appl. No.: 19/329,961

(22) Filed: Sep. 16, 2025

Related U.S. Application Data
(63) Continuation of application No. PCT/CN2024/081840, filed on Mar. 15, 2024.

(30) Foreign Application Priority Data
Mar. 17, 2023 (WO) PCT/CN2023/082099

(51) Int. Cl. C07K 16/28

(52) U.S. Cl. CPC C07K 16/2827; C07K 2317/24; C07K 2317/31; C07K 2317/41; C07K 2317/565; C07K 2317/569; C07K 2317/92

(57) ABSTRACT

The present disclosure provides for antibodies or antigen-binding fragments thereof that bind to human 4Ig-B7H3.

### CROSS REFERENCE TO RELATED APPLICATIONS

This application is a continuation of International Patent Application No. PCT/CN2024/081840, filed Mar. 15, 2024, which claims the benefit of priority from International Patent Application No. PCT/CN2023/082099, filed Mar. 17, 2023, entitled “Anti-B7H3 Antibodies and Methods of Use,” which are hereby incorporated by reference in their entirety.

### SEQUENCE LISTING

The present application contains a sequence listing which has been submitted electronically in xml format and is hereby incorporated by reference in its entirety. Said xml file was created on Mar. 14, 2024, is 139,799 bytes in size, and named 138881-0836_PCT.xml.

### FIELD OF THE DISCLOSURE

The present disclosure is directed to antibodies and antigen-binding fragments that specifically bind to human 4Ig-B7H3.

### BACKGROUND

B7H3 or B7-H3 is a type I transmembrane protein identified in 2001 from a dendritic cell (DC) cDNA library (Chapoval, Ni et al. 2001). It is also called CD276, B7RP-2, etc. B7H3, as part of the B7 immunoregulatory family, includes two isoforms, 4Ig-B7H3 and 2Ig-B7H3, in human, and 4Ig-B7H3 is the main isoform expressed in malignant cells (Steinberger, Majdic et al. 2004). The extracellular domain of mouse B7H3 is composed of a single pair of immunoglobulin variable (IgV)-like and immunoglobulin constant (IgC)-like domains, whereas human B7H3 contains single pair or two identical pairs due to exon duplication (Sun, Richards et al. 2002, Steinberger, Majdic et al. 2004). The intracellular domain of B7H3 is short and has no known signaling motif (Picarda, Ohaegbulam et al. 2016). Besides the transmembrane form, B7H3 may exist in a soluble form, which is cleaved from membrane B7H3 by proteinase or caused by alternative splicing (Zhang, Hou et al. 2008).

Belonging to the B7 protein family, B7H3 shares 20-27% amino-acid identity with other B7 family ligands, but the receptor(s) remain to be elucidated (Chapoval, Ni et al. 2001). To date, the molecular mechanisms by which B7H3 participates in immune evasion remain elusive and B7H3 function in T cell-mediated adaptive immunity is still controversial. Although originally identified as a T cell co-stimulatory molecule (Chapoval, Ni et al. 2001, Zhang, Chen et al. 2004), more later studies suggest it mainly plays immune suppressive role (Suh, Gajewska et al. 2003, Prasad, Nguyen et al. 2004, Fukushima, Sumi et al. 2007, Veenstra, Flynn et al. 2015). Meanwhile, the crystal structure of mouse B7H3 reveals that the FG Loop is important for mB7H3-mediated inhibition of T cell proliferation (Vigdorovich, Ramagopal et al. 2013). Notably, non-small cell lung cancer (NSCLC) with high B7H3 expression was associated with a lower number of tumor-infiltrating lymphocytes and was more refractory to anti-PD1 therapy, suggesting a role for B7H3 in immune evasion. Therefore, B7H3 targeted therapy combined with anti-PD-1/PD-L1 antibody therapy is a promising approach for B7H3-expressing NSCLCs (Altan, Pelekanou et al. 2017).

Besides immune regulation, B7H3 also has intrinsic protumor function. TCGA analysis revealed that B7H3 expression is correlated with the EGFR/PI3K/AKT pathway, MAPK pathway, and EMT process across cancer types, and aberrant B7H3 expression promotes tumor metastasis, angiogenesis, glycolytic metabolism, and drug resistance (Tekle, Nygren et al. 2012, Liu, Zhang et al. 2015, Lee, Martin-Orozco et al. 2017, Flem-Karlsen, Fodstad et al. 2018, Liu, Zhang et al. 2019, Lai, Sun et al. 2020). Accordingly, B7H3 overexpression is correlated with poor prognosis and bad clinical outcomes in many cancer types (Crispen, Sheinin et al. 2008, Bachawal, Jensen et al. 2015, Fan, Zhu et al. 2016, Song, Shi et al. 2016, Wu, Zhao et al. 2016, Benzon, Zhao et al. 2017).

Numerous studies have reported that B7H3 is highly overexpressed on a wide range of solid tumors and tumor vasculature but has a limited expression at low level in normal tissues (Loo, Alderson et al. 2012, Seaman, Zhu et al. 2017, Du, Hirabayashi et al. 2019, Yamato, Hasegawa et al. 2022), making it an attractive target for cancer therapy. The majority of the B7H3 targeting assets are in the early development stage. Y-Mabs has developed omburtamab with radionuclides including I131 or Lu177, and the lead asset is I131-omburtamab to target brain tumors. Enoblituzumab is a Fc-enhanced B7H3 antibody in phase I/II, while limited anti-tumor efficacy was demonstrated in anti-PD1/PD-L1 refractory patients when combined with an anti-PD1 antibody. B7H3 ADCs, including MGC018 from MacroGenics and DS-7300 from Daiichi Sankyo, displayed preliminary anti-tumor activity in phase I studies and well-tolerated safety profiles.

There are no approved therapeutic antibodies against B7H3, and there remains an unmet medical need for therapeutics targeting B7H3.

### SUMMARY OF THE DISCLOSURE

The present disclosure is directed to antibodies and antigen-binding fragments thereof that specifically bind human 4Ig-B7H3.

In embodiments, the present disclosure is directed to an antibody or antigen-binding fragment thereof that specifically binds human 4Ig-B7H3, comprising:
(1) a heavy chain variable region (VH) that comprises (a) a HCDR1 (Heavy Chain Complementarity Determining Region 1) of SEQ ID NO: 7, (b) a HCDR2 of SEQ ID NO: 8, and (c) a HCDR3 of SEQ ID NO: 9; or
(2) a heavy chain variable region that comprises (a) a HCDR1 of SEQ ID NO: 12, (b) a HCDR2 of SEQ ID NO: 13, and (c) a HCDR3 of SEQ ID NO: 14.

In embodiments, the antibody or antigen-binding fragment thereof comprises:
(1) a heavy chain variable region comprising an amino acid sequence at least 90, 91, 92, 93, 94, 95, 96, 97, 98, or 99% identical to SEQ ID NO: 10;
(2) a heavy chain variable region comprising an amino acid sequence at least 90, 91, 92, 93, 94, 95, 96, 97, 98, or 99% identical to SEQ ID NO: 20;
(3) a heavy chain variable region comprising an amino acid sequence at least 90, 91, 92, 93, 94, 95, 96, 97, 98, or 99% identical to SEQ ID NO: 24;
(4) a heavy chain variable region comprising an amino acid sequence at least 90, 91, 92, 93, 94, 95, 96, 97, 98, or 99% identical to SEQ ID NO: 26;
(5) a heavy chain variable region comprising an amino acid sequence at least 90, 91, 92, 93, 94, 95, 96, 97, 98, or 99% identical to SEQ ID NO: 30;
(6) a heavy chain variable region comprising an amino acid sequence at least 90, 91, 92, 93, 94, 95, 96, 97, 98, or 99% identical to SEQ ID NO: 32;
(7) a heavy chain variable region comprising an amino acid sequence at least 90, 91, 92, 93, 94, 95, 96, 97, 98, or 99% identical to SEQ ID NO: 34;
(8) a heavy chain variable region comprising an amino acid sequence at least 90, 91, 92, 93, 94, 95, 96, 97, 98, or 99% identical to SEQ ID NO: 36;
(9) a heavy chain variable region comprising an amino acid sequence at least 90, 91, 92, 93, 94, 95, 96, 97, 98, or 99% identical to SEQ ID NO: 40;
(10) a heavy chain variable region comprising an amino acid sequence at least 90, 91, 92, 93, 94, 95, 96, 97, 98, or 99% identical to SEQ ID NO: 42;
(11) a heavy chain variable region comprising an amino acid sequence at least 90, 91, 92, 93, 94, 95, 96, 97, 98, or 99% identical to SEQ ID NO: 44;
(12) a heavy chain variable region comprising an amino acid sequence at least 90, 91, 92, 93, 94, 95, 96, 97, 98, or 99% identical to SEQ ID NO: 46;
(13) a heavy chain variable region comprising an amino acid sequence at least 90, 91, 92, 93, 94, 95, 96, 97, 98, or 99% identical to SEQ ID NO: 48;
(14) a heavy chain variable region comprising an amino acid sequence at least 90, 91, 92, 93, 94, 95, 96, 97, 98, or 99% identical to SEQ ID NO: 50;
(15) a heavy chain variable region comprising an amino acid sequence at least 90, 91, 92, 93, 94, 95, 96, 97, 98, or 99% identical to SEQ ID NO: 15;
(16) a heavy chain variable region comprising an amino acid sequence at least 90, 91, 92, 93, 94, 95, 96, 97, 98, or 99% identical to SEQ ID NO: 52;
(17) a heavy chain variable region comprising an amino acid sequence at least 90, 91, 92, 93, 94, 95, 96, 97, 98, or 99% identical to SEQ ID NO: 54; or
(18) a heavy chain variable region comprising an amino acid sequence at least 90, 91, 92, 93, 94, 95, 96, 97, 98, or 99% identical to SEQ ID NO: 56.

In embodiments, in the antibody or antigen-binding fragment thereof, one, two, three, four, five, six, seven, eight, nine, or ten amino acids within at least one of SEQ ID NO: 10, SEQ ID NO: 20, SEQ ID NO: 24, SEQ ID NO: 26, SEQ ID NO: 30, SEQ ID NO: 32, SEQ ID NO: 34, SEQ ID NO: 36, SEQ ID NO: 40, SEQ ID NO: 42, SEQ ID NO: 44, SEQ ID NO: 46, SEQ ID NO: 48, SEQ ID NO: 50, SEQ ID NO: 15, SEQ ID NO: 52, SEQ ID NO: 54, and SEQ ID NO: 56 have been inserted, deleted, or substituted.

In embodiments, the antibody or antigen-binding fragment thereof comprises:
(1) a heavy chain variable region comprising SEQ ID NO: 10;
(2) a heavy chain variable region comprising SEQ ID NO: 20;
(3) a heavy chain variable region comprising SEQ ID NO: 24;
(4) a heavy chain variable region comprising SEQ ID NO: 26;
(5) a heavy chain variable region comprising SEQ ID NO: 30;
(6) a heavy chain variable region comprising SEQ ID NO: 32;
(7) a heavy chain variable region comprising SEQ ID NO: 34;
(8) a heavy chain variable region comprising SEQ ID NO: 36;
(9) a heavy chain variable region comprising SEQ ID NO: 40;
(10) a heavy chain variable region comprising SEQ ID NO: 42;
(11) a heavy chain variable region comprising SEQ ID NO: 44;
(12) a heavy chain variable region comprising SEQ ID NO: 46;
(13) a heavy chain variable region comprising SEQ ID NO: 48;
(14) a heavy chain variable region comprising SEQ ID NO: 50;
(15) a heavy chain variable region comprising SEQ ID NO: 15;
(16) a heavy chain variable region comprising SEQ ID NO: 52;
(17) a heavy chain variable region comprising SEQ ID NO: 54; or
(18) a heavy chain variable region comprising SEQ ID NO: 56.

In embodiments, the antibody or antigen-binding fragment thereof is a monoclonal antibody, a chimeric antibody, a humanized antibody, a human engineered antibody, a single chain antibody (scFv), a Fab fragment, a Fab′ fragment, a F(ab′)2 fragment, a heavy chain antibody (HcAb), or a VHH.

In embodiments, a multi-specific antibody or antigen-binding fragment thereof comprises at least a first antigen binding domain that specifically binds a first human tumor antigen (TAA), wherein the first TAA is human 4Ig-B7H3 and the first antigen binding domain comprises the antibody or antigen-binding fragment thereof disclosed herein; and at least a second antigen binding domain that specifically binds a second human TAA.

In embodiments, the multi-specific antibody or antigen-binding fragment thereof is a bispecific antibody.

In embodiments, the multi-specific antibody or antigen-binding fragment thereof comprises an amino acid linker, and the amino acid linker is any sequence of SEQ ID NO: 79 to SEQ ID NO: 121.

In embodiments, the antibody or antigen-binding fragment thereof comprises a heavy chain constant region of the subclass of IgG1, IgG2, IgG3, or IgG4, and/or a light chain constant region of the type of kappa or lambda.

In embodiments, the antibody or antigen-binding fragment thereof comprises a heavy chain constant region of the subclass of IgG1.

In embodiments, the antibody or antigen-binding fragment thereof has antibody dependent cellular cytotoxicity (ADCC) or complement dependent cytotoxicity (CDC).

In embodiments, the antibody or antigen-binding fragment thereof has reduced glycosylation or no glycosylation or is hypofucosylated.

In embodiments, the antibody or antigen-binding fragment thereof comprises increased bisecting GlcNac structures.

The present disclosure is also directed to a VHH, wherein said VHH specifically binds to human 4Ig-B7H3.

In embodiments, the VHH comprises:
(1) a heavy chain variable region (VH) that comprises (a) a HCDR1 (Heavy Chain Complementarity Determining Region 1) of SEQ ID NO: 7, (b) a HCDR2 of SEQ ID NO: 8, and (c) a HCDR3 of SEQ ID NO: 9; or
(2) a heavy chain variable region that comprises (a) a HCDR1 of SEQ ID NO: 12, (b) a HCDR2 of SEQ ID NO: 13, and (c) a HCDR3 of SEQ ID NO: 14.

In embodiments, the VHH comprises:
(1) a heavy chain variable region comprising an amino acid sequence at least 90, 91, 92, 93, 94, 95, 96, 97, 98, or 99% identical to SEQ ID NO: 10;
(2) a heavy chain variable region comprising an amino acid sequence at least 90, 91, 92, 93, 94, 95, 96, 97, 98, or 99% identical to SEQ ID NO: 20;
(3) a heavy chain variable region comprising an amino acid sequence at least 90, 91, 92, 93, 94, 95, 96, 97, 98, or 99% identical to SEQ ID NO: 24;
(4) a heavy chain variable region comprising an amino acid sequence at least 90, 91, 92, 93, 94, 95, 96, 97, 98, or 99% identical to SEQ ID NO: 26;
(5) a heavy chain variable region comprising an amino acid sequence at least 90, 91, 92, 93, 94, 95, 96, 97, 98, or 99% identical to SEQ ID NO: 30;
(6) a heavy chain variable region comprising an amino acid sequence at least 90, 91, 92, 93, 94, 95, 96, 97, 98, or 99% identical to SEQ ID NO: 32;
(7) a heavy chain variable region comprising an amino acid sequence at least 90, 91, 92, 93, 94, 95, 96, 97, 98, or 99% identical to SEQ ID NO: 34;
(8) a heavy chain variable region comprising an amino acid sequence at least 90, 91, 92, 93, 94, 95, 96, 97, 98, or 99% identical to SEQ ID NO: 36;
(9) a heavy chain variable region comprising an amino acid sequence at least 90, 91, 92, 93, 94, 95, 96, 97, 98, or 99% identical to SEQ ID NO: 40;
(10) a heavy chain variable region comprising an amino acid sequence at least 90, 91, 92, 93, 94, 95, 96, 97, 98, or 99% identical to SEQ ID NO: 42;
(11) a heavy chain variable region comprising an amino acid sequence at least 90, 91, 92, 93, 94, 95, 96, 97, 98, or 99% identical to SEQ ID NO: 44;
(12) a heavy chain variable region comprising an amino acid sequence at least 90, 91, 92, 93, 94, 95, 96, 97, 98, or 99% identical to SEQ ID NO: 46;
(13) a heavy chain variable region comprising an amino acid sequence at least 90, 91, 92, 93, 94, 95, 96, 97, 98, or 99% identical to SEQ ID NO: 48;
(14) a heavy chain variable region comprising an amino acid sequence at least 90, 91, 92, 93, 94, 95, 96, 97, 98, or 99% identical to SEQ ID NO: 50;
(15) a heavy chain variable region comprising an amino acid sequence at least 90, 91, 92, 93, 94, 95, 96, 97, 98, or 99% identical to SEQ ID NO: 15;
(16) a heavy chain variable region comprising an amino acid sequence at least 90, 91, 92, 93, 94, 95, 96, 97, 98, or 99% identical to SEQ ID NO: 52;
(17) a heavy chain variable region comprising an amino acid sequence at least 90, 91, 92, 93, 94, 95, 96, 97, 98, or 99% identical to SEQ ID NO: 54; or
(18) a heavy chain variable region comprising an amino acid sequence at least 90, 91, 92, 93, 94, 95, 96, 97, 98, or 99% identical to SEQ ID NO: 56.

In embodiments, in the VHH, one, two, three, four, five, six, seven, eight, nine, or ten amino acids within at least one of SEQ ID NO: 10, SEQ ID NO: 20, SEQ ID NO: 24, SEQ ID NO: 26, SEQ ID NO: 30, SEQ ID NO: 32, SEQ ID NO: 34, SEQ ID NO: 36, SEQ ID NO: 40, SEQ ID NO: 42, SEQ ID NO: 44, SEQ ID NO: 46, SEQ ID NO: 48, SEQ ID NO: 50, SEQ ID NO: 15, SEQ ID NO: 52, SEQ ID NO: 54, and SEQ ID NO: 56 have been inserted, deleted, or substituted.

In embodiments, the VHH comprises:
(1) a heavy chain variable region comprising SEQ ID NO: 10;
(2) a heavy chain variable region comprising SEQ ID NO: 20;
(3) a heavy chain variable region comprising SEQ ID NO: 24;
(4) a heavy chain variable region comprising SEQ ID NO: 26;
(5) a heavy chain variable region comprising SEQ ID NO: 30;
(6) a heavy chain variable region comprising SEQ ID NO: 32;
(7) a heavy chain variable region comprising SEQ ID NO: 34;
(8) a heavy chain variable region comprising SEQ ID NO: 36;
(9) a heavy chain variable region comprising SEQ ID NO: 40;
(10) a heavy chain variable region comprising SEQ ID NO: 42;
(11) a heavy chain variable region comprising SEQ ID NO: 44;
(12) a heavy chain variable region comprising SEQ ID NO: 46;
(13) a heavy chain variable region comprising SEQ ID NO: 48;
(14) a heavy chain variable region comprising SEQ ID NO: 50;
(15) a heavy chain variable region comprising SEQ ID NO: 15;
(16) a heavy chain variable region comprising SEQ ID NO: 52;
(17) a heavy chain variable region comprising SEQ ID NO: 54; or
(18) a heavy chain variable region comprising SEQ ID NO: 56.

In embodiments, a multi-specific antibody or antigen-binding fragment thereof comprises at least a first antigen binding domain that specifically binds a first human tumor antigen (TAA), wherein the first TAA is human 4Ig-B7H3, and the first antigen binding domain comprises the VHH disclosed herein; and at least a second antigen binding domain that specifically binds a second human TAA.

In embodiments, the multi-specific antibody or antigen-binding fragment thereof is a bispecific antibody.

In embodiments, the multi-specific antibody or antigen-binding fragment thereof comprises an amino acid linker, and the amino acid linker is any sequence of SEQ ID NO: 79 to SEQ ID NO: 121.

In embodiments, a pharmaceutical composition comprises the antibody or antigen-binding fragment thereof, or VHH, disclosed herein and a pharmaceutically acceptable carrier.

In embodiments, the present disclosure is directed to an isolated nucleic acid that encodes the antibody or antigen-binding fragment thereof disclosed herein.

In embodiments, the present disclosure is directed to a vector comprising the nucleic acid disclosed herein.

In embodiments, the present disclosure is directed to a host cell comprising the nucleic acid or the vector disclosed herein.

In embodiments, the present disclosure is directed to a process for producing an antibody or antigen-binding fragment thereof comprising cultivating the host cell and recovering the antibody or antibody fragment disclosed herein from the culture.

In embodiments, the anti-human 4Ig-B7H3 antibodies or antigen-binding fragments thereof have at least one of the following features:
(1) specific binding and high affinity for human 4Ig-B7H3;
(2) being a humanized antibody having low immunogenicity risk to human; and
(3) good purity and being easy to purify.

In embodiments, the present disclosure provides anti-human 4Ig-B7H3 VHHs that are highly humanized and have low immunogenicity risk to humans, while maintaining specific binding and high affinity to human 4Ig-B7H3, and/or exhibiting high purity and being easy to purify.

### BRIEF DESCRIPTION OF THE DRAWINGS

FIG. 1 shows the binding of BGA-026 and BGA-056 to B7H3 antigen by ELISA.

FIG. 2 shows the binding of BGA-026 and BGA-056 to HEK293 cells by flow cytometry.

FIG. 3 shows the binding of BGA-026 chimeric and BGA-2042 to H358 cells by flow cytometry.

FIG. 4 shows the binding of BGA-056 chimeric and BGA-174 to H358 cells by flow cytometry.

FIGS. 5A-5B show the binding of VHH1_C001 and VHH1_C002 to B7H3-positive cancer cell line H358 (FIG. 5A) and B7H3-negative cancer cell line MDA-MB-453 (FIG. 5B) by flow cytometry.

FIGS. 6A-6D show the binding of VHH1_C001 and VHH1_C002 to 4-Ig-B7H3 and 2Ig-B7H3 by surface plasmon resonance (SPR).

FIG. 7 shows the epitope binning of VHH1_C001 and VHH1_C002.

### DETAILED DESCRIPTION

The present disclosure is directed to anti-human 4Ig-B7H3 antibodies and antigen-binding fragments thereof. The disclosed antibodies have desirable binding affinities and other desirable attributes, such as low immunogenicity risk to humans. The anti-human 4Ig-B7H3 antibodies and antibody fragments thereof may be used to construct multi-specific antibodies with additional functionalities, such as binding a second human tumor associated antigen (TAA), immune checkpoint inhibition, or immune stimulation, to construct antibody drug conjugates (ADC), or to fuse with other domains to form fusion proteins. Furthermore, the anti-4Ig-B7H3 antibodies and the constructs thereof, or pharmaceutical compositions comprising the same, could be used for treating 4Ig-B7H3-expressing cancers and associated disorders.

### Definitions

Unless specifically defined below or elsewhere in this document, all other technical and scientific terms used herein have the meaning commonly understood by one of ordinary skill in the art.

As used herein, including in the appended claims, the singular forms of words such as “a,” “an,” and “the” include their corresponding plural references unless the context clearly dictates otherwise.

The term “or” is used to mean, and is used interchangeably with, the term “and/or” unless the context clearly dictates otherwise.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within one or more than one standard deviation per the practice in the art. “About” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” should be assumed to be within an acceptable error range for that particular value or composition.

The term “human 4Ig-B7H3” refers to the 4Ig isoform of a type I transmembrane protein B7H3 in human. The nucleic acid sequence of human 4Ig-B7H3 is set forth in SEQ ID NO: 1, based on GenBank sequence Accession No: NM_001024736.1. The amino acid sequence of human 4Ig-B7H3 is SEQ ID NO: 2.

The terms “administration,” and “administering,” as used herein, when applied to an animal, human, subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell.

The term “subject” or “patient” herein includes any organism, preferably an animal, more preferably a mammal (e.g., rat, mouse, dog, cat, rabbit, primate) and most preferably a human (e.g., a patient comprising, or at risk of having, a disorder described herein).

“Treating” any disease or disorder refers in one aspect to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically (e.g., stabilization of a discernible symptom), physiologically (e.g., stabilization of a physical parameter), or both.

The term “affinity” as used herein refers to the strength of interaction between antibody and antigen. Within the antigen, the variable regions of the antibody interact through non-covalent forces with the antigen at numerous sites. In general, the more interactions, the stronger the affinity.

The term “antibody,” (“Ab”), as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL or Vκ) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997); Lefranc, M.-P., The Immunologist, 7, 132-136 (1999); Lefranc, M.-P. et al., Dev. Comp. Immunol., 27, 55-77 (2003)).

The term “antibody” includes, but is not limited to, monoclonal antibodies, human antibodies, humanized antibodies, chimeric antibodies, anti-idiotypic (anti-Id) antibodies, and human engineered antibodies. The antibodies can be of any isotype/class (e.g., IgG, IgE, IgM, IgD, IgA and IgY), or subclass (e.g., IgG1, IgG2, IgG3, IgG4, IgA1 and IgA2).

The term “chimeric antibody” means molecules made up of domains from different species, i.e., fusing the variable domain of an antibody from one host species (e.g. mouse, rabbit, llama, etc.) with the constant domain of an antibody from a different species (e.g. human).

In some embodiments, the anti-4Ig-B7H3 antibodies comprise at least one antigen-binding site, which may be at least a variable region. In some embodiments, the anti-4Ig-B7H3 antibodies comprise an antigen-binding fragment from an 4Ig-B7H3 antibody described herein. In some embodiments, the anti-4Ig-B7H3 antibody is isolated or recombinant. In some embodiments, the anti-4Ig-B7H3 antibodies also encompass multi-specific antibodies targeting 4Ig-B7H3 as at least one arm and targeting other antigen(s) as another arm(s).

The term “monoclonal antibody” or “mAb” or “Mab” herein means a population of substantially homogeneous antibodies, i.e., the antibody molecules in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively.

Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (FR3), CDR-3 (CDR3), and FR-4 (FR4). The positions of the CDRs and framework regions can be determined using various well known definitions in the art, e.g., Kabat, Chothia, AbM, and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997) ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2), and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2, and LCDR3 in the light chain variable domain and HCDR1, HCDR2, and HCDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but are not limited to, Fab, Fab′, F(ab′)2, and Fv fragments; diabodies; linear antibodies; single-chain antibody molecules, e.g., single chain Fv (ScFv); nanobodies (or VHH antibody); multi-specific antibodies formed from antibody fragments; and bicyclic peptides (Hurov, K. et al., 2021. Journal for ImmunoTherapy of Cancer, 9(11)).

As used herein, an antibody or antigen-binding antibody fragment “specifically binds” to an antigen (e.g., a protein), meaning the antibody exhibits preferential binding to that target as compared to other proteins, but this specificity does not require absolute binding specificity. A “specific” or “selective” binding reaction is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten times when compared to the background level of binding and does not specifically bind in a significant amount to other antigens present in the sample.

“Antigen-binding domain” as used herein, comprises at least six CDRs (or three CDRs in terms of single domain antibody) and specifically binds to an epitope. An “antigen-binding domain” of a multi-specific antibody (e.g., a bispecific antibody) comprises a first antigen binding domain that specifically binds to a first epitope and a second antigen binding domain also comprised of at least three CDRs that specifically binds to a second epitope. Multi-specific antibodies can be bispecific, trispecific, tetraspecific, etc., with antigen binding domains directed to each specific epitope. Multi-specific antibodies can be multivalent (e.g., a bispecific tetravalent antibody) that comprises multiple antigen binding domains, for example, 2, 3, 4, or more antigen binding domains that specifically bind to a first epitope and 2, 3, 4, or more antigen binding domains that specifically bind a second epitope. An “antigen-binding domain” of a single chain antibody, e.g., a heavy chain antibody, or a VHH, comprises an antigen binding domain that specifically binds to an epitope without pairing with an additional variable domain. The binding site of an immunoglobulin single variable domain is formed by a single VH/VHH or VL domain.

The terms “VHH domain,” “VHH antibody,” “VHH antibody fragment,” and “VHH” (also known as a nanobody), as used herein, refer to that which was originally described as the antigen binding domain of heavy chain-only antibodies produced by camelids (Naturally occurring antibodies devoid of light chains, C. Hamers-Casterman et al., Nature, volume 363, pages 446-448 (1993)) and is distinguished from the heavy chain variable domains (VH domain) of a conventional tetramer antibody. The VHH antibody retains the immunoglobulin fold of conventional four-chain antibodies, with only three hypervariable loops—CDR1, CDR2, and CDR3—to bind to its target. Many VHHs bind to their targets with affinities similar to conventional full-size antibodies, and may possess other properties superior to conventional full-size antibodies.

The term “human antibody” herein means an antibody that comprises only human immunoglobulin protein sequences. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine, rabbit, llama, etc.) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental (e.g., rodent) antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable region, or other combinations of sequences or subsequences. Sequence identity can be determined using the methods described herein, for example, aligning two sequences using BLAST, ALIGN, or another alignment algorithm known in the art. The corresponding human germline nucleic acid or amino acid sequence can have at least about 90%, 91%, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant” or “KD” or “M” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, 10⁻¹¹ M, 10⁻¹² M, or 10⁻¹³ M.

The term “cancer” or “tumor” used herein has the broadest meaning as understood in the art and refers to the physiological condition in mammals that is typically characterized by unregulated cell growth. In the context of the present disclosure, the cancer is not limited to a certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g., its binding affinity to 4Ig-B7H3. Common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H}I interfaces or VH/VL interfaces of antibodies (see, e.g., US 2011/0287009, US2007/0178552, WO 96/027011, WO 98/050431, and Zhu et al., 1997, Protein Science, 6:781-788). In some embodiments, knob-into-holes ensure the correct pairing of two different heavy chains together during the manufacture of multi-specific antibodies. For example, multi-specific antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also ensure correct pairing.

Examples of algorithms that are suitable for determining percent sequence identity and sequence similarity are the BLAST algorithms, which are described in Altschul et al, Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89: 10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4: 11-17, (1988), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include anti-4Ig-B7H3 antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination components.

The term “combination therapy” refers to the administration of two or more therapeutic agents to treat a therapeutic condition or disorder. Such administration encompasses co-administration of these therapeutic agents in a substantially simultaneous manner. Such administration also encompasses co-administration in multiple or in separate containers or formulations (e.g., capsules, powders, and liquids) for each active ingredient. Powders and/or liquids can be reconstituted or diluted to a desired dose prior to administration. In addition, “combination therapy” encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-4IG-B7H3 antibody is administered to the subject at the same time as, just before, or just after administration of an additional therapeutic agent. In certain embodiments, an anti-4Ig-B7H3 antibody is administered as a co-formulation with an additional therapeutic agent.

### Anti-4Ig-B7H3 Antibodies

TABLE 1
Sequences of anti-4Ig-B7H3 antibodies

| Antibody | SEQ ID NO | | Sequence |
|---|---|---|---|
| BGA-026 | SEQ ID NO: 59 | HCDR1 | PRTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 62 | HCDR1 | PRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 10 | VHH AA | EVQLVESGGGLVQTGDSLRLSCAASPRTFQTYFMG |
| | | | WFRQAPGKEREFVSVINWSGGTSYYKDSVKGRFTI |
| | | | SRDNAKNTVYLQMNNLKPEDTAVYYCAAKGSGTY |
| | | | DREGEYDYWGQGTQVTVSS |
| | SEQ ID NO: 19 | VHH DNA | GAAGTGCAACTGGTGGAGTCCGGCGGCGGGCTG |
| | | | GTCCAAACCGGGGACAGCCTGAGACTGAGCTGC |
| | | | GCCGCCTCCCCTAGAACCTTTCAGACCTACTTCAT |
| | | | GGGCTGGTTCAGACAAGCCCCCGGCAAGGAGAG |
| | | | AGAGTTCGTGAGCGTGATCAACTGGAGCGGCGG |
| | | | CACAAGCTACTACAAGGACAGCGTGAAGGGCAG |
| | | | ATTCACCATCAGCAGAGACAACGCCAAGAACAC |
| | | | AGTGTACCTGCAGATGAACAACCTGAAGCCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCAAGTGACCGTGAGCA |
| | | | GC |
| BGA-154 | SEQ ID NO: 59 | HCDR1 | PRTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 62 | HCDR1 | PRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 20 | VHH AA | EVQLVESGGGLVQPGGSLRLSCAASPRTFQTYFMG |
| | | | WFRQAPGKEREFVSVINWSGGTSYYKDSVKGRFTI |
| | | | SRDNSKNTVYLQMNSLRAEDTAVYYCAAKGSGTY |
| | | | DREGEYDYWGQGTLVTVSS |
| | SEQ ID NO: 21 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTCAGACCTACTTCAT |
| | | | GGGCTGGTTCAGACAAGCCCCCGGCAAGGAGAG |
| | | | AGAGTTCGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CGTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-1541 | SEQ ID NO: 64 | HCDR1 | GRTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 65 | HCDR1 | GRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 22 | VHH AA | EVQLVESGGGLVQPGGSLRLSCAASGRTFQTYFMG |
| | | | WFRQAPGKEREFVSVINWSGGTSYYKDSVKGRFTI |
| | | | SRDNSKNTVYLQMNSLRAEDTAVYYCAAKGSGTY |
| | | | DREGEYDYWGQGTLVTVSS |
| | SEQ ID NO: 23 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCGGCAGAACCTTTCAGACCTACTTCA |
| | | | TGGGCTGGTTCAGACAAGCCCCCGGCAAGGAGA |
| | | | GAGAGTTCGTGAGCGTGATCAACTGGAGCGGCG |
| | | | GCACAAGCTACTACAAGGACAGCGTGAAGGGCA |
| | | | GATTCACCATCAGCAGAGACAACAGCAAGAACA |
| | | | CCGTGTACCTGCAGATGAACAGCCTGAGAGCCGA |
| | | | GGACACCGCCGTGTACTACTGCGCCGCCAAGGGC |
| | | | AGCGGCACCTACGACAGAGAGGGCGAGTACGAC |
| | | | TACTGGGGCCAAGGCACCCTGGTGACCGTGAGC |
| | | | AGC |
| BGA-1542 | SEQ ID NO: 66 | HCDR1 | PFTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 67 | HCDR1 | PFTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 24 | VHH AA | EVQLVESGGGLVQPGGSLRLSCAASPFTFQTYFMG |
| | | | WFRQAPGKEREFVSVINWSGGTSYYKDSVKGRFTI |
| | | | SRDNSKNTVYLQMNSLRAEDTAVYYCAAKGSGTY |
| | | | DREGEYDYWGQGTLVTVSS |
| | SEQ ID NO: 25 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTTTCACCTTTCAGACCTACTTCAT |
| | | | GGGCTGGTTCAGACAAGCCCCCGGCAAGGAGAG |
| | | | AGAGTTCGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CGTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-1543 | SEQ ID NO: 68 | HCDR1 | PRTFSTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 69 | HCDR1 | PRTFSTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 26 | VHH AA | EVQLVESGGGLVQPGGSLRLSCAASPRTFSTYFMG |
| | | | WFRQAPGKEREFVSVINWSGGTSYYKDSVKGRFTI |
| | | | SRDNSKNTVYLQMNSLRAEDTAVYYCAAKGSGTY |
| | | | DREGEYDYWGQGTLVTVSS |
| | SEQ ID NO: 27 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTAGCACCTACTTCAT |
| | | | GGGCTGGTTCAGACAAGCCCCCGGCAAGGAGAG |
| | | | AGAGTTCGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CGTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-1544 | SEQ ID NO: 59 | HCDR1 | PRTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 62 | HCDR1 | PRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 28 | VHH AA | EVQLVESGGGLVQPGGSLRLSCAASPRTFQTYFMG |
| | | | WVRQAPGKEREFVSVINWSGGTSYYKDSVKGRFTI |
| | | | SRDNSKNTVYLQMNSLRAEDTAVYYCAAKGSGTY |
| | | | DREGEYDYWGQGTLVTVSS |
| | SEQ ID NO: 29 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTCAGACCTACTTCAT |
| | | | GGGCTGGGTGAGACAAGCCCCCGGCAAGGAGAG |
| | | | AGAGTTCGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CGTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-1545 | SEQ ID NO: 59 | HCDR1 | PRTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 62 | HCDR1 | PRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 30 | VHH AA | EVQLVESGGGLVQPGGSLRLSCAASPRTFQTYFMG |
| | | | WFRQAPGKGREFVSVINWSGGTSYYKDSVKGRFTI |
| | | | SRDNSKNTVYLQMNSLRAEDTAVYYCAAKGSGTY |
| | | | DREGEYDYWGQGTLVTVSS |
| | SEQ ID NO: 31 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTCAGACCTACTTCAT |
| | | | GGGCTGGTTCAGACAAGCCCCCGGCAAGGGCAG |
| | | | AGAGTTCGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CGTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-1546 | SEQ ID NO: 59 | HCDR1 | PRTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 62 | HCDR1 | PRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 32 | VHH AA | EVQLVESGGGLVQPGGSLRLSCAASPRTFQTYFMG |
| | | | WFRQAPGKELEFVSVINWSGGTSYYKDSVKGRFTI |
| | | | SRDNSKNTVYLQMNSLRAEDTAVYYCAAKGSGTY |
| | | | DREGEYDYWGQGTLVTVSS |
| | SEQ ID NO: 33 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTCAGACCTACTTCAT |
| | | | GGGCTGGTTCAGACAAGCCCCCGGCAAGGAGCT |
| | | | GGAGTTCGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CGTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-1547 | SEQ ID NO: 59 | HCDR1 | PRTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 62 | HCDR1 | PRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 34 | VHH AA | EVQLVESGGGLVQPGGSLRLSCAASPRTFQTYFMG |
| | | | WFRQAPGKEREWVSVINWSGGTSYYKDSVKGRFT |
| | | | ISRDNSKNTVYLQMNSLRAEDTAVYYCAAKGSGTY |
| | | | DREGEYDYWGQGTLVTVSS |
| | SEQ ID NO: 35 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTCAGACCTACTTCAT |
| | | | GGGCTGGTTCAGACAAGCCCCCGGCAAGGAGAG |
| | | | AGAGTGGGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CGTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-1548 | SEQ ID NO: 59 | HCDR1 | PRTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 62 | HCDR1 | PRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 36 | VHH AA | EVQLVESGGGLVQPGGSLRLSCAASPRTFQTYFMG |
| | | | WFRQAPGKEREFVSVINWSGGTSYYKDSVKGRFTI |
| | | | SRDNSKNTLYLQMNSLRAEDTAVYYCAAKGSGTY |
| | | | DREGEYDYWGQGTLVTVSS |
| | SEQ ID NO: 37 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTCAGACCTACTTCAT |
| | | | GGGCTGGTTCAGACAAGCCCCCGGCAAGGAGAG |
| | | | AGAGTTCGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CCTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-1549 | SEQ ID NO: 59 | HCDR1 | PRTFQTYF |
| | | (IMGT) | |
| | SEQ ID NO: 60 | HCDR2 | INWSGGTS |
| | | (IMGT) | |
| | SEQ ID NO: 70 | HCDR3 | AKKGSGTYDREGEYDY |
| | | (IMGT) | |
| | SEQ ID NO: 62 | HCDR1 | PRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 38 | VHH AA | EVQLVESGGGLVQPGGSLRLSCAASPRTFQTYFMG |
| | | | WFRQAPGKEREFVSVINWSGGTSYYKDSVKGRFTI |
| | | | SRDNSKNTVYLQMNSLRAEDTAVYYCAKKGSGTY |
| | | | DREGEYDYWGQGTLVTVSS |
| | SEQ ID NO: 39 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTCAGACCTACTTCAT |
| | | | GGGCTGGTTCAGACAAGCCCCCGGCAAGGAGAG |
| | | | AGAGTTCGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CGTGTACCTGCAGATGAACAGCCTGAGAGCCGAG |
| | | | GACACCGCCGTGTACTACTGCGCCAAGAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-2041 | SEQ ID NO: 59 | HCDR1 | PRTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 62 | HCDR1 | PRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 40 | VHH AA | EVQLVESGGGLVQPGGSLRLSCAASPRTFQTYFMG |
| | | | WFRQAPGKGLEWVSVINWSGGTSYYKDSVKGRFT |
| | | | ISRDNSKNTLYLQMNSLRAEDTAVYYCAAKGSGTY |
| | | | DREGEYDYWGQGTLVTVSS |
| | SEQ ID NO: 41 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTCAGACCTACTTCAT |
| | | | GGGCTGGTTCAGACAAGCCCCCGGCAAGGGCCT |
| | | | GGAGTGGGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CCTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-2042 | SEQ ID NO: 59 | HCDR1 | PRTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 62 | HCDR1 | PRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 42 | VHH AA | EVQLVESGGGLVQPGGSLRLSCAASPRTFQTYFMG |
| | | | WFRQAPGKELEWVSVINWSGGTSYYKDSVKGRFTI |
| | | | SRDNSKNTLYLQMNSLRAEDTAVYYCAAKGSGTY |
| | | | DREGEYDYWGQGTLVTVSS |
| | SEQ ID NO: 43 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTCAGACCTACTTCAT |
| | | | GGGCTGGTTCAGACAAGCCCCCGGCAAGGAGCT |
| | | | GGAGTGGGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CCTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-2043 | SEQ ID NO: 66 | HCDR1 | PFTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 67 | HCDR1 | PFTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 44 | VHH AA | EVQLVESGGGLVQPGGSLRLSCAASPFTFQTYFMG |
| | | | WFRQAPGKELEWVSVINWSGGTSYYKDSVKGRFTI |
| | | | SRDNSKNTLYLQMNSLRAEDTAVYYCAAKGSGTY |
| | | | DREGEYDYWGQGTLVTVSS |
| | SEQ ID NO: 45 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTTTCACCTTTCAGACCTACTTCAT |
| | | | GGGCTGGTTCAGACAAGCCCCCGGCAAGGAGCT |
| | | | GGAGTGGGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CCTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-2044 | SEQ ID NO: 68 | HCDR1 | PRTFSTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 69 | HCDR1 | PRTFSTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 46 | VHH AA | EVQLVESGGGLVQPGGSLRLSCAASPRTFSTYFMG |
| | | | WFRQAPGKELEWVSVINWSGGTSYYKDSVKGRFTI |
| | | | SRDNSKNTLYLQMNSLRAEDTAVYYCAAKGSGTY |
| | | | DREGEYDYWGQGTLVTVSS |
| | SEQ ID NO: 47 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTAGCACCTACTTCAT |
| | | | GGGCTGGTTCAGACAAGCCCCCGGCAAGGAGCT |
| | | | GGAGTGGGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CCTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-2045 | SEQ ID NO: 71 | HCDR1 | PFTFSTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 72 | HCDR1 | PFTFSTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 48 | VHH AA | EVQLVESGGGLVQPGGSLRLSCAASPFTFSTYFMG |
| | | | WFRQAPGKELEWVSVINWSGGTSYYKDSVKGRFTI |
| | | | SRDNSKNTLYLQMNSLRAEDTAVYYCAAKGSGTY |
| | | | DREGEYDYWGQGTLVTVSS |
| | SEQ ID NO: 49 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTTTCACCTTTAGCACCTACTTCAT |
| | | | GGGCTGGTTCAGACAAGCCCCCGGCAAGGAGCT |
| | | | GGAGTGGGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CCTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |
| BGA-2046 | SEQ ID NO: 59 | HCDR1 | PRTFQTYF |
| | | (IMGT) | |
| | SEQ ID NO: 60 | HCDR2 | INWSGGTS |
| | | (IMGT) | |
| | SEQ ID NO: 61 | HCDR3 | AAKGSGTYDREGEYDY |
| | | (IMGT) | |
| | SEQ ID NO: 62 | HCDR1 | PRTFQTY |
| | | (Chothia) | |
| | SEQ ID NO: 63 | HCDR2 | NWSGGT |
| | | (Chothia) | |
| | SEQ ID NO: 9 | HCDR3 | KGSGTYDREGEYDY |
| | | (Chothia) | |
| | SEQ ID NO: 7 | HCDR1 | TYFMG |
| | | (Kabat) | |
| | SEQ ID NO: 8 | HCDR2 | VINWSGGTSYYKDSVKG |
| | | (Kabat) | |
| | SEQ ID NO: 9 | HCDR3 | KGSGTYDREGEYDY |
| | | (Kabat) | |
| | SEQ ID NO: 50 | VHH AA | EVQLVESGGGLVQPGGSLRLSCAASPRTFQTYFMG |
| | | | WFRQAPGKGREWVSVINWSGGTSYYKDSVKGRFT |
| | | | ISRDNSKNTLYLQMNSLRAEDTAVYYCAAKGSGTY |
| | | | DREGEYDYWGQGTLVTVSS |
| | SEQ ID NO: 51 | VHH DNA | GAGGTGCAACTCGTGGAGAGCGGCGGGGGCCTG |
| | | | GTCCAACCCGGGGGCAGCCTGAGACTGAGCTGC |
| | | | GCCGCTAGCCCTAGAACCTTTCAGACCTACTTCAT |
| | | | GGGCTGGTTCAGACAAGCCCCCGGCAAGGGCAG |
| | | | AGAGTGGGTGAGCGTGATCAACTGGAGCGGCGG |
| | | | CACAAGCTACTACAAGGACAGCGTGAAGGGCAG |
| | | | ATTCACCATCAGCAGAGACAACAGCAAGAACAC |
| | | | CCTGTACCTGCAGATGAACAGCCTGAGAGCCGAG |
| | | | GACACCGCCGTGTACTACTGCGCCGCCAAGGGCA |
| | | | GCGGCACCTACGACAGAGAGGGCGAGTACGACT |
| | | | ACTGGGGCCAAGGCACCCTGGTGACCGTGAGCA |
| | | | GC |

TABLE 2
Sequences of anti-4Ig-B7H3 antibodies

| Antibody | SEQ ID NO | | Sequence |
|---|---|---|---|
| BGA-056 | SEQ ID NO: 73 | HCDR1 | GFTFSDYH |
| | | (IMGT) | |
| | SEQ ID NO: 74 | HCDR2 | ILNTGLSP |
| | | (IMGT) | |
| | SEQ ID NO: 75 | HCDR3 | AQGYYRATPSQAN |
| | | (IMGT) | |
| | SEQ ID NO: 76 | HCDR1 | GFTFSDY |
| | | (Chothia) | |
| | SEQ ID NO: 77 | HCDR2 | LNTGLS |
| | | (Chothia) | |
| | SEQ ID NO: 14 | HCDR3 | GYYRATPSQAN |
| | | (Chothia) | |
| | SEQ ID NO: 12 | CDR1 | DYHMS |
| | | (Kabat) | |
| | SEQ ID NO: 13 | CDR2 | GILNTGLSPTYAATVTG |
| | | (Kabat) | |
| | SEQ ID NO: 14 | CDR3 | GYYRATPSQAN |
| | | (Kabat) | |
| | SEQ ID NO: 15 | VHH AA | EVQLLESGGGLVQPGGSLRLSCAASGFTFSDYHMS |
| | | | WVRQAPGKGLEWVSGILNTGLSPTYAATVTGRFTIS |
| | | | RDNAKNTLYLSMNSLKPEDTAVYYCAQGYYRATPS |
| | | | QANRGQGTQVTVSS |
| | SEQ ID NO: 122 | VHH DNA | GAGGTGCAACTGCTGGAGTCCGGCGGGGGCCTGG |
| | | | TGCAGCCCGGCGGGAGCCTGAGACTGAGCTGCGC |
| | | | CGCTAGCGGCTTCACCTTCAGCGACTACCACATGA |
| | | | GCTGGGTGAGACAAGCCCCCGGCAAGGGCCTGG |
| | | | AGTGGGTGAGCGGCATCCTGAACACCGGCCTGAG |
| | | | CCCCACCTACGCCGCCACCGTGACCGGCAGATTC |
| | | | ACCATCAGCAGAGATAACGCCAAAAACACCCTGT |
| | | | ACCTGAGCATGAACAGCCTGAAGCCCGAGGACAC |
| | | | CGCCGTGTACTACTGCGCCCAAGGCTACTACAGAG |
| | | | CCACCCCTAGCCAAGCCAACAGAGGCCAAGGCAC |
| | | | CCAAGTGACCGTGAGCAGC |
| BGA-174 | SEQ ID NO: 76 | HCDR1 | GFTFSDY |
| | | (IMGT) | |
| | SEQ ID NO: 74 | HCDR2 | ILNTGLSP |
| | | (IMGT) | |
| | SEQ ID NO: 75 | HCDR3 | AQGYYRATPSQAN |
| | | (IMGT) | |
| | SEQ ID NO: 76 | HCDR1 | GFTFSDY |
| | | (Chothia) | |
| | SEQ ID NO: 77 | HCDR2 | LNTGLS |
| | | (Chothia) | |
| | SEQ ID NO: 14 | HCDR3 | GYYRATPSQAN |
| | | (Chothia) | |
| | SEQ ID NO: 12 | HCDR1 | DYHMS |
| | | (Kabat) | |
| | SEQ ID NO: 13 | HCDR2 | GILNTGLSPTYAATVTG |
| | | (Kabat) | |
| | SEQ ID NO: 14 | HCDR3 | GYYRATPSQAN |
| | | (Kabat) | |
| | SEQ ID NO: 52 | VHH AA | EVQLLESGGGLVQPGGSLRLSCAASGFTFSDYHMS |
| | | | WVRQAPGKGLEWVSGILNTGLSPTYAATVTGRFTIS |
| | | | RDNSKNTLYLQMNSLRAEDTAVYYCAQGYYRATPS |
| | | | QANRGQGTLVTVSS |
| | SEQ ID NO: 53 | VHH DNA | GAAGTGCAGCTGCTGGAATCCGGGGGGGGGCTCG |
| | | | TGCAGCCCGGGGGCAGCCTGAGACTGAGCTGCGC |
| | | | CGCTAGCGGCTTCACCTTCAGCGACTACCACATGA |
| | | | GCTGGGTGAGACAAGCCCCCGGCAAGGGCCTGG |
| | | | AGTGGGTGAGCGGCATCCTGAACACCGGCCTGAG |
| | | | CCCCACCTACGCCGCCACCGTGACCGGCAGATTC |
| | | | ACCATCAGCAGAGACAACAGCAAGAACACCCTGT |
| | | | ACCTGCAGATGAACAGCCTGAGAGCCGAGGACAC |
| | | | CGCCGTGTACTACTGCGCCCAAGGCTACTACAGAG |
| | | | CCACCCCTAGCCAAGCCAACAGAGGCCAAGGCAC |
| | | | CCTGGTGACCGTGAGCAGC |
| BGA-1741 | SEQ ID NO: 73 | HCDR1 | GFTFSDYH |
| | | (IMGT) | |
| | SEQ ID NO: 74 | HCDR2 | ILNTGLSP |
| | | (IMGT) | |
| | SEQ ID NO: 78 | HCDR3 | AKGYYRATPSQAN |
| | | (IMGT) | |
| | SEQ ID NO: 76 | HCDR1 | GFTFSDY |
| | | (Chothia) | |
| | SEQ ID NO: 77 | HCDR2 | LNTGLS |
| | | (Chothia) | |
| | SEQ ID NO: 14 | HCDR3 | GYYRATPSQAN |
| | | (Chothia) | |
| | SEQ ID NO: 12 | HCDR1 | DYHMS |
| | | (Kabat) | |
| | SEQ ID NO: 13 | HCDR2 | GILNTGLSPTYAATVTG |
| | | (Kabat) | |
| | SEQ ID NO: 14 | HCDR3 | GYYRATPSQAN |
| | | (Kabat) | |
| | SEQ ID NO: 54 | VHH AA | EVQLLESGGGLVQPGGSLRLSCAASGFTFSDYHMS |
| | | | WVRQAPGKGLEWVSGILNTGLSPTYAATVTGRFTIS |
| | | | RDNSKNTLYLQMNSLRAEDTAVYYCAKGYYRATPS |
| | | | QANRGQGTLVTVSS |
| | SEQ ID NO: 55 | VHH DNA | GAAGTGCAGCTGCTGGAATCCGGGGGGGGGCTCG |
| | | | TGCAGCCCGGGGGCAGCCTGAGACTGAGCTGCGC |
| | | | CGCTAGCGGCTTCACCTTCAGCGACTACCACATGA |
| | | | GCTGGGTGAGACAAGCCCCCGGCAAGGGCCTGG |
| | | | AGTGGGTGAGCGGCATCCTGAACACCGGCCTGAG |
| | | | CCCCACCTACGCCGCCACCGTGACCGGCAGATTC |
| | | | ACCATCAGCAGAGACAACAGCAAGAACACCCTGT |
| | | | ACCTGCAGATGAACAGCCTGAGAGCCGAGGACAC |
| | | | CGCCGTGTACTACTGCGCCAAGGGCTACTACAGA |
| | | | GCCACCCCTAGCCAAGCCAACAGAGGCCAAGGCA |
| | | | CCCTGGTGACCGTGAGCAGC |
| BGA-1742 | SEQ ID NO: 73 | HCDR1 | GFTFSDYH |
| | | (IMGT) | |
| | SEQ ID NO: 74 | HCDR2 | ILNTGLSP |
| | | (IMGT) | |
| | SEQ ID NO: 75 | HCDR3 | AQGYYRATPSQAN |
| | | (IMGT) | |
| | SEQ ID NO: 76 | HCDR1 | GFTFSDY |
| | | (Chothia) | |
| | SEQ ID NO: 77 | HCDR2 | LNTGLS |
| | | (Chothia) | |
| | SEQ ID NO: 14 | HCDR3 | GYYRATPSQAN |
| | | (Chothia) | |
| | SEQ ID NO: 12 | HCDR1 | DYHMS |
| | | (Kabat) | |
| | SEQ ID NO: 13 | HCDR2 | GILNTGLSPTYAATVTG |
| | | (Kabat) | |
| | SEQ ID NO: 14 | HCDR3 | GYYRATPSQAN |
| | | (Kabat) | |
| | SEQ ID NO: 56 | VHH AA | EVQLLESGGGLVQPGGSLRLSCAASGFTFSDYHMS |
| | | | WVRQAPGKGLEWVSGILNTGLSPTYAATVTGRFTIS |
| | | | RDNSKNTLYLQMNSLRAEDTAVYYCAQGYYRATPS |
| | | | QANWGQGTLVTVSS |
| | SEQ ID NO: 57 | VHH DNA | GAAGTGCAGCTGCTGGAATCCGGGGGGGGGCTCG |
| | | | TGCAGCCCGGGGGCAGCCTGAGACTGAGCTGCGC |
| | | | CGCTAGCGGCTTCACCTTCAGCGACTACCACATGA |
| | | | GCTGGGTGAGACAAGCCCCCGGCAAGGGCCTGG |
| | | | AGTGGGTGAGCGGCATCCTGAACACCGGCCTGAG |
| | | | CCCCACCTACGCCGCCACCGTGACCGGCAGATTC |
| | | | ACCATCAGCAGAGACAACAGCAAGAACACCCTGT |
| | | | ACCTGCAGATGAACAGCCTGAGAGCCGAGGACAC |
| | | | CGCCGTGTACTACTGCGCCCAAGGCTACTACAGAG |
| | | | CCACCCCTAGCCAAGCCAACTGGGGCCAAGGCAC |
| | | | CCTGGTGACCGTGAGCAGC |

The present disclosure provides for antibodies or antigen-binding fragments thereof that specifically bind to human 4Ig-B7H3 (i.e., anti-human 4Ig-B7H3 antibodies or antigen-binding fragments thereof). The antibodies or antigen-binding fragments may be produced as described below.

In embodiments, the anti-human 4Ig-B7H3 antibodies or antibody fragments (e.g., antigen-binding fragments) include a heavy chain variable region (VH) domain having an amino acid sequence of at least one of SEQ ID NO: 10, SEQ ID NO: 20, SEQ ID NO: 24, SEQ ID NO: 26, SEQ ID NO: 30, SEQ ID NO: 32, SEQ ID NO: 34, SEQ ID NO: 36, SEQ ID NO: 40, SEQ ID NO: 42, SEQ ID NO: 44, SEQ ID NO: 46, SEQ ID NO: 48, SEQ ID NO: 50, SEQ ID NO: 15, SEQ ID NO: 52, SEQ ID NO: 54, or SEQ ID NO: 56 (Tables 1-2). In embodiments, the anti-human 4Ig-B7H3 antibodies or antigen-binding fragments comprise a heavy chain complementarity determining region (HCDR) having an amino acid sequence of any one of the HCDRs listed in Tables 1-2. In one aspect, the anti-human 4Ig-B7H3 antibodies or antigen-binding fragments include one, two, three, or more HCDRs having an amino acid sequence of any of the HCDRs listed in Tables 1-2.

In one embodiment, the antibodies or an antigen-binding fragments thereof that specifically bind to human 4Ig-B7H3 include one or more complementarity determining regions (CDRs) comprising an amino acid sequence selected from SEQ ID NO: 7, SEQ ID NO: 8, and SEQ ID NO: 9; or selected from SEQ ID NO: 12, SEQ ID NO: 13, and SEQ ID NO: 14.

In one embodiment, the anti-human 4Ig-B7H3 antibodies or antigen-binding fragments thereof comprise: (i) a HCDR1, a HCDR2, and a HCDR3 from the VH set forth in SEQ ID NO: 10, SEQ ID NO: 20, SEQ ID NO: 24, SEQ ID NO: 26, SEQ ID NO: 30, SEQ ID NO: 32, SEQ ID NO: 34, SEQ ID NO: 36, SEQ ID NO: 40, SEQ ID NO: 42, SEQ ID NO: 44, SEQ ID NO: 46, SEQ ID NO: 48, SEQ ID NO: 50, SEQ ID NO: 15, SEQ ID NO: 52, SEQ ID NO: 54, or SEQ ID NO: 56.

In another embodiment, the antibodies or an antigen-binding fragments thereof that specifically bind to human 4Ig-B7H3 comprise: (a) a heavy chain variable region comprising three HCDRs which are HCDR1 comprising an amino acid sequence of SEQ ID NO: 7, HCDR2 comprising an amino acid sequence of SEQ ID NO: 8, and HCDR3 comprising an amino acid sequence of SEQ ID NO: 9, according to the Kabat numbering.

In another embodiment, the antibodies or an antigen-binding fragments thereof that specifically bind to human 4Ig-B7H3 comprise: (a) a heavy chain variable region comprising three HCDRs which are HCDR1 comprising an amino acid sequence of SEQ ID NO: 12, HCDR2 comprising an amino acid sequence of SEQ ID NO: 13, and HCDR3 comprising an amino acid sequence of SEQ ID NO: 14, according to the Kabat numbering.

In one embodiment, the antibodies or an antigen-binding fragments thereof of the present disclosure comprise: (a) a heavy chain variable region comprising an amino acid sequence of at least one of SEQ ID NO: 10, SEQ ID NO: 20, SEQ ID NO: 24, SEQ ID NO: 26, SEQ ID NO: 30, SEQ ID NO: 32, SEQ ID NO: 34, SEQ ID NO: 36, SEQ ID NO: 40, SEQ ID NO: 42, SEQ ID NO: 44, SEQ ID NO: 46, SEQ ID NO: 48, SEQ ID NO: 50, SEQ ID NO: 15, SEQ ID NO: 52, SEQ ID NO: 54, or SEQ ID NO: 56, or an amino acid sequence at least 90%, 91%, 92%, 93%, 94%, 95%, 96%, 97%, 98%, or 99% identical to any one or more of SEQ ID NO: 10, SEQ ID NO: 20, SEQ ID NO: 24, SEQ ID NO: 26, SEQ ID NO: 30, SEQ ID NO: 32, SEQ ID NO: 34, SEQ ID NO: 36, SEQ ID NO: 40, SEQ ID NO: 42, SEQ ID NO: 44, SEQ ID NO: 46, SEQ ID NO: 48, SEQ ID NO: 50, SEQ ID NO: 15, SEQ ID NO: 52, SEQ ID NO: 54, and SEQ ID NO: 56.

Other antibodies or antigen-binding fragments thereof of the present disclosure include amino acids that have been changed, yet have at least 60%, 70%, 80%, 90%, 91%, 92%, 93%, 94%, 95% 96%, 97%, 98%, or 99% percent identity in the CDR regions compared with the CDR regions disclosed in Tables 1-2. In some aspects, the CDR regions include amino acid changes (insertion, deletion, or substitution, which may be conservative amino acid substitutions) wherein no more than 1, 2, 3, 4, or 5 amino acids have been changed in the CDR regions when compared with the CDR regions disclosed in Tables 1-2, while maintaining binding specificity and affinity.

Other antibodies or antigen-binding fragments thereof of the present disclosure include those in which the amino acids or nucleic acids encoding the amino acids have been changed in the variable regions (e.g., the frameworks regions of the variable regions), yet have at least 60, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% percent identity to the sequences of the variable regions disclosed in Tables 1-2, while retaining binding specificity/affinity. In some embodiments, the corresponding sequences of CDRs do not change. In some aspects, no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids have been changed in the variable regions (e.g., the frameworks regions of the variable regions) when compared with the variable regions disclosed in Tables 1-2, while the antibodies or antigen-binding fragments thereof retain binding specificity/affinity. In some embodiments, the corresponding sequences of CDRs do not change.

In some embodiments, the present disclosure provides for an antibody or antigen-binding fragment thereof that specifically binds human 4Ig-B7H3 and includes a VH that comprises (a) a HCDR1 of SEQ ID NO: 7, (b) a HCDR2 of SEQ ID NO: 8, and (c) a HCDR3 of SEQ ID NO: 9, and the amino acids P26, F37, and A94 in the framework region have been retained.

In some embodiments, the present disclosure provides for anti-human 4Ig-B7H3 antibodies or antigen-binding fragments thereof disclosed in Table 1, or a variant thereof, wherein the HCDR1, HCDR2, and HCDR3 are not changed and amino acids P26, F37, and A94 are retained.

In some embodiments, the present disclosure provides for an antibodies or antigen-binding fragments thereof that specifically bind human 4Ig-B7H3 and include a heavy chain variable region comprising an amino acid sequence at least 90, 91, 92, 93, 94, 95, 96, 97, 98, or 99% identical to at least one of SEQ ID NO: 10, SEQ ID NO: 20, SEQ ID NO: 24, SEQ ID NO: 26, SEQ ID NO: 30, SEQ ID NO: 32, SEQ ID NO: 34, SEQ ID NO: 36, SEQ ID NO: 40, SEQ ID NO: 42, SEQ ID NO: 44, SEQ ID NO: 46, SEQ ID NO: 48, or SEQ ID NO: 50, wherein the amino acids P26, F37, and A94 in the framework region are retained.

In another embodiment, the present disclosure provides for antibodies or antigen-binding fragments thereof that specifically bind to human 4Ig-B7H3 with a binding affinity (K_{D}) of from 1×10⁻⁶ M to 1×10⁻¹¹ M. In another embodiment, the anti-4Ig-B7H3 antibodies or antigen-binding fragments thereof bind to human 4Ig-B7H3 with a binding affinity (K_{D}) of about 1×10⁻⁶ M, about 1×10⁻⁷ M, about 1×10⁻⁸ M, about 1×10⁻⁹ M, about 1×10⁻¹⁰ M, or about 1×10⁻¹¹ M.

The present disclosure also provides nucleic acid sequences that encode VH or VL of the antibodies that specifically bind to human 4Ig-B7H3. Such nucleic acid sequences may be optimized for expression in mammalian cells.

The present disclosure also provides for antibodies and antigen-binding fragments thereof that bind to the same epitope as do the anti-4Ig-B7H3 antibodies described in Tables 1-2. Additional antibodies and antigen-binding fragments thereof can therefore be identified based on their ability to cross-compete (e.g., to competitively inhibit the binding of, in a statistically significant manner) with the antibodies described in Tables 1-2 in binding assays. The ability of a test antibody to inhibit the binding of antibodies and antigen-binding fragments thereof of the present disclosure to 4Ig-B7H3 demonstrates that the test antibody can compete with that antibody or antigen-binding fragments thereof for binding to 4Ig-B7H3. Such an antibody can, without being bound to any one theory, bind to the same or a related (e.g., a structurally similar or spatially proximal) epitope on 4Ig-B7H3 as the antibody or antigen-binding fragments thereof with which it competes. In a certain aspect, the antibody that binds to the same epitope on 4Ig-B7H3 as the antibodies or antigen-binding fragments thereof of the present disclosure is a human or humanized monoclonal antibody. Such human or humanized monoclonal antibodies can be prepared and isolated as described herein.

In some embodiments, the antibody or antigen-binding fragment thereof is a monoclonal antibody, a chimeric antibody, a humanized antibody, a human engineered antibody, a single chain antibody (scFv), a Fab fragment, a Fab′ fragment, a F(ab′)2 fragment, or a VHH.

### Anti-4Ig-B7H3 Multi-Specific Antibodies

In one embodiment, the anti-4Ig-B7H3 antibodies or antigen-binding fragments thereof disclosed herein can be used to construct multi-specific antibodies with additional functionalities, such as binding a second human tumor associated antigen (TAA), immune checkpoint inhibition, or immune stimulation. In some examples, 4Ig-B7H3 functions as a first TAA.

In one embodiment, the 4Ig-B7H3 antibodies or antigen binding fragments disclosed herein can be incorporated into an anti-4Ig-B7H3×TAA multi-specific antibody, wherein TAA is an antibody or fragment thereof directed to any human tumor associated antigen (TAA). An antibody is a multi-specific antibody molecule if, for example, it comprises a number of antigen binding domains, wherein at least one antigen binding domain sequence specifically binds 4Ig-B7H3 and a second antigen binding domain sequence specifically binds a TAA. In embodiments, the multi-specific antibody comprises a third, fourth, or fifth antigen binding domain. In embodiments, the multi-specific antibody is a bispecific antibody, a tri-specific antibody, or tetra-specific antibody. In each embodiment, the multi-specific antibody comprises at least one anti-4Ig-B7H3 antigen binding domain and at least one anti-TAA antigen binding domain.

In one embodiment, the multi-specific antibody is a bispecific antibody. As used herein, a bispecific antibody specifically binds only two antigens. The bispecific antibody comprises a first antigen binding domain that specifically binds human 4Ig-B7H3 and a second antigen binding domain that specifically binds a second TAA. Included is a bispecific antibody comprising a second heavy chain variable domain and a second light chain variable domain that specifically binds a second TAA and a first heavy chain variable domain and/or a first light chain variable domain that specifically binds human 4Ig-B7H3. In another embodiment, the bispecific antibody comprises an antigen binding fragment of an antibody that specifically binds 4Ig-B7H3 and an antigen binding fragment that specifically binds a TAA. When the bispecific antibody comprises antigen binding fragments, the antigen-binding fragment can be a Fab, F(ab′)₂, Fv, a single chain Fv (scFv), or a single domain antibody.

In embodiments, the multi-specific antibody of the present disclosure binds to a second human TAA and/or human 4Ig-B7H3 with a binding affinity (K_{D}) of from 1×10⁻⁶ M to 1×10⁻¹¹ M. In embodiments, the multi-specific antibody of the present disclosure binds to a second human TAA and/or human 4Ig-B7H3 with a binding affinity (K_{D}) of about 1×10⁻⁶ M, about 1×10⁻⁷ M, about 1×10⁻⁸ M, about 1×10⁻⁹ M, about 1×10⁻¹⁰ M, or about 1×10⁻¹¹ M.

In one embodiment, the present disclosure provides for a multi-specific antibody or antigen-binding fragment thereof, wherein the first antigen binding domain that specifically binds to human 4Ig-B7H3 comprises the anti-human 4Ig-B7H3 antibody or antigen-binding fragment thereof described herein and the second antigen binding domain specifically binds to a second human TAA.

In another embodiment, the present disclosure provides for a multi-specific antibody or antigen-binding fragment thereof, wherein the first antigen binding domain that specifically binds to human 4Ig-B7H3 comprises at least one of SEQ ID NO: 10, SEQ ID NO: 20, SEQ ID NO: 24, SEQ ID NO: 26, SEQ ID NO: 30, SEQ ID NO: 32, SEQ ID NO: 34, SEQ ID NO: 36, SEQ ID NO: 40, SEQ ID NO: 42, SEQ ID NO: 44, SEQ ID NO: 46, SEQ ID NO: 48, SEQ ID NO: 50, SEQ ID NO: 15, SEQ ID NO: 52, SEQ ID NO: 54, or SEQ ID NO: 56, and the second antigen binding domain specifically binds to a second human TAA.

Previous experimentation (Coloma and Morrison, Nature Biotech. 15: 159-163 (1997)) described a tetravalent bispecific antibody that was engineered by fusing DNA encoding a single chain anti-dansyl antibody Fv (scFv) after the C terminus (CH3-scFv) or after the hinge (hinge-scFv) of an IgG3 anti-dansyl antibody. The present disclosure provides multivalent antibodies (e.g. tetravalent antibodies) with at least two antigen binding domains, which can be readily produced by recombinant expression of nucleic acid encoding the polypeptide chains of the antibody. The multivalent antibody herein comprises three to eight, but preferably four, antigen binding domains that specifically bind at least two antigens.

In one embodiment, the multi-specific antibody is a bispecific antibody. In another embodiment, the multi-specific antibody further comprises an amino acid linker described herein, such as any one of SEQ ID NO: 79 to SEQ ID NO: 121.

Anti-Human 4Ig-B7H3 Antibodies Conjugated with a Cytotoxin

The presently disclosed anti-human 4Ig-B7H3 antibodies or antigen-binding fragments thereof can be used to construct antibody drug conjugates (ADC). In one embodiment, the antibody or antigen-binding fragment thereof is conjugated to a cytotoxin. In another embodiment, the antibody or antigen-binding fragment thereof is conjugated to a cytotoxin via a cytotoxin linker.

### Cytotoxin

Cytotoxins or cytotoxic agents include any agent that is detrimental to the growth, viability, or propagation of cells, including, but not limited to, tubulin-interacting agents and DNA-damaging agents. Examples of suitable cytotoxic agents and chemotherapeutic agents that can be conjugated to the antibodies of the present disclosure include, e.g., 1-(2chloroethyl)-1,2-dimethanesulfonyl hydrazide, 1,8-dihydroxy-bicyclo[7.3.1]trideca-4,9-diene-2,6-diyne-13-one, 1-dehydrotestosterone, 5-fluorouracil, 6-mercaptopurine, 6-thioguanine, 9-amino camptothecin, actinomycin D, amanitins, aminopterin, anguidine, anthracycline, anthramycin (AMC), auristatins, bleomycin, busulfan, butyric acid, calicheamicins (e.g., calicheamicin gamma1), camptothecin, carminomycins, carmustine, cemadotins, cisplatin, colchicin, combretastatins, cyclophosphamide, cytarabine, cytochalasin B, dactinomycin, daunorubicin, decarbazine, diacetoxypentyldoxorubicin, dibromomannitol, dihydroxy anthracin dione, disorazoles, dolastatin (e.g., dolastatin 10), doxorubicin, duocarmycin, echinomycins, eleutherobins, emetine, epothilones, esperamicin, estramustines, ethidium bromide, etoposide, fluorouracils, geldanamycins, gramicidin D, glucocorticoids, irinotecans, kinesin spindle protein (KSP) inhibitors, leptomycins, leurosines, lidocaine, lomustine (CCNU), maytansinoids, mechlorethamine, melphalan, mercatopurines, methopterins, methotrexate, mithramycin, mitomycin, mitoxantrone, N8-acetyl spermidine, podophyllotoxins, procaine, propranolol, pteridines, puromycin, pyrrolobenzodiazepines (PBDs), rhizoxins, streptozotocin, tallysomycins, taxol, tenoposide, tetracaine, thioepa chlorambucil, tomaymycins, topotecans, tubulysin, vinblastine, vincristine, vindesine, vinorelbines, and derivatives of any of the foregoing.

### Cytotoxin Linkers

Cytotoxin linkers or linkers for ADC are any group or moiety that links, connects, or bonds the antibody or antigen-binding fragments described herein with a therapeutic moiety, e.g., a cytotoxic agent. Suitable linkers may be found, for example, in Antibody-Drug Conjugates and Immunotoxins; Phillips, G. L, Ed.; Springer Verlag: New York, 2013; Antibody-Drug Conjugates; Ducry, L, Ed.; Humana Press, 2013; Antibody-Drug Conjugates; Wang, J., Shen, W.-C, and Zaro, J. L, Eds.; Springer International Publishing, 2015, the contents of each of which is incorporated herein by reference in their entireties.

Generally, suitable binding agents or cytotoxin linkers for the antibody conjugates described herein are those that are sufficiently stable to exploit the circulating half-life of the antibody and, at the same time, capable of releasing the payload after antigen-mediated internalization of the conjugate. Linkers can be cleavable or non-cleavable. Cleavable linkers include linkers that are cleaved by intracellular metabolism following internalization, e.g., cleavage via hydrolysis, reduction, or enzymatic reaction. Non-cleavable linkers include linkers that release an attached payload via lysosomal degradation of the antibody following internalization. Suitable linkers include, but are not limited to, acid-labile linkers, hydrolysis-labile linkers, enzymatically cleavable linkers, reduction-labile linkers, self-immolative linkers, and non-cleavable linkers. Suitable linkers also include, but are not limited to, those that are or comprise peptides, glucuronides, succinimide-thioethers, polyethylene glycol (PEG) units, hydrazones, mal-caproyl units, dipeptide units, valine-citruline units, and para-aminobenzyl (PAB) units.

Any cytotoxin linker molecule or linker technology known in the art can be used to create or construct an ADC of the present disclosure. In certain embodiments, the cytotoxin linker is a cleavable linker. According to other embodiments, the linker is a non-cleavable linker. Exemplary linkers that can be used in the context of the present disclosure include linkers that comprise or consist of e.g., GGFG, MC (6-maleimidocaproyl), MP (maleimidopropanoyl), val-cit (valine-citrulline), val-ala (valine-alanine) dipeptide site in protease-cleavable linker, ala-phe (alanine-phenylalanine) dipeptide site in protease-cleavable linker, PAB (p-aminobenzyloxycarbonyl), SPP (N-succinimidyl 4-(2-pyridylthio) pentanoate), SMCC (N-succinimidyl 4-(N-maleimidomethyl)cyclohexane-1 carboxylate), SIAB (N-succinimidyl (4-iodo-acetyl)aminobenzoate), and variants and combinations thereof. Additional examples of linkers that can be used in the context of the present disclosure are provided, e.g., in U.S. Pat. No. 7,754,681 and in Ducry, Bioconjugate Chem., 2010, 27:5-13, and the references cited therein, the contents of which are incorporated by reference herein in their entireties.

In certain embodiments, the cytotoxin linkers are stable in physiological conditions. In certain embodiments, the linkers are cleavable, for instance, able to release at least the payload portion in the presence of an enzyme or at a particular pH range or value. In some embodiments, a linker comprises an enzyme-cleavable moiety. Illustrative enzyme-cleavable moieties include, but are not limited to, peptide bonds, ester linkages, hydrazones, and disulfide linkages. In some embodiments, the linker comprises a cathepsin-cleavable linker.

In some embodiments, the cytotoxin linker comprises a non-cleavable moiety.

Suitable cytotoxin linkers also include, but are not limited to, those that are chemically bonded to two cysteine residues of a single binding agent, e.g., antibody. Such linkers can serve to mimic the antibody's disulfide bonds that are disrupted as a result of the conjugation process.

In some embodiments, the cytotoxin linker comprises one or more amino acids. Suitable amino acids include natural, non-natural, standard, non-standard, proteinogenic, non-proteinogenic, and L- or D-amino acids. In some embodiments, the cytotoxin linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or combination thereof. In some embodiments, the linker comprises valine and citrulline. In some embodiments, the cytotoxin linker comprises lysine, valine, and citrulline. In some embodiments, the linker comprises lysine, valine, and alanine. In some embodiments, the linker comprises valine and alanine.

### Fusion Protein Targeting Human 4Ig-B7H3

The anti-human 4Ig-B7H3 antibodies or antigen-binding fragments thereof disclosed herein can be fused with other proteins or other functional domains to form fusion proteins or chimeric proteins.

In some embodiments, the anti-human 4Ig-B7H3 antibodies or antigen-binding fragments thereof are fused with immune checkpoints, immune stimulators, cytokines, or a second TAA either directly or indirectly via an amino acid linker described herein.

In one embodiment, the anti-human 4Ig-B7H3 antibodies or antigen-binding fragments thereof are fused with functional domains or receptor subunits that might serve to transduce the signal from the scFv/VHH and confer antibody specificity to immune cells, such as T cells or NK cells as well as other effector cells.

In some embodiments, the anti-human 4Ig-B7H3 antibodies or antigen-binding fragments thereof are used to a construct chimeric antigen receptors (CAR). Detailed information about CAR constructs may be found in Guedan et al., Mol Ther Methods Clin Dev 2019; 12:145-156.

The functional domains or receptor subunits comprise, for example, transmembrane domains, hinge regions, intracellular signaling domains, and co-stimulatory domains.

### Constant Region and Fc Region Modifications

The heavy chain constant region could be the wild type sequences of heavy chain constant region from the subclass of IgG1, IgG2, IgG3, or IgG4. The light chain constant region could be the wild type sequences of light chain from kappa or lambda type. In one embodiment, the heavy chain constant region is the wild type sequence of constant region from IgG1. The light chain constant region is the wild type sequence of light chain from kappa chain. In one embodiment, the heavy chain constant region has the amino acid sequences of SEQ ID NO: 4 or SEQ ID NO: 18. In another embodiment, the heavy chain constant region comprises mutations E233P, L234A, L235A, G236del, and P329A.

In one embodiment, the Fc region could be wild type Fc region of the subclass of IgG1, IgG2, IgG3, or IgG4. In one embodiment, the antibody or antigen-binding fragment thereof comprises a Fc domain of IgG1 or IgG4 with reduced effector function.

In one embodiment, the antibody or antigen-binding fragment thereof comprises a Fc domain with extended half-life. In another embodiment, the antibody or antigen-binding fragment thereof comprises a Fc domain of IgG1, wherein YTE mutation (M252Y/S254T/T256E, EU numbering, as described in U.S. Pat. No. 7,658,921) located at CH2 of IgG Fc region were introduced.

In another embodiment, antibodies of the present disclosure have strong Fc-mediated effector functions, and the antibodies mediate antibody-dependent cellular cytotoxicity (ADCC) against target cells.

In aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcθRI, FcγRII, FcγRIII, and FcRn have been mapped and variants with improved binding have been described (see Shields et al., J. Biol. Chem. 276:6591-6604, 2001).

In still another aspect, the glycosylation of the antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for an antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures, which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G L, et al., 2010 MAbs, 2:181-189). However, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of an antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel S M, et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al, 2000 Nature Medicine, 6:443-446; Arnold J. N., 2007 Annu Rev immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate antibodies with low ADCC and CDC but with good stability, it is possible to modify the hinge and Fc region of human IgG4 and introduce a number of alterations. These modified IgG4 Fc molecules can be found in SEQ ID NOs: 83-88, U.S. Pat. No. 8,735,553 to Li et al.

In another embodiment, the antibodies of the present disclosure comprise an Fc domain of human IgG4 with S228P and/or R409K substitutions (according to EU numbering system).

### Amino Acid Linkers

The domains and/or regions of the polypeptide chains of the bispecific tetravalent antibodies or constructs disclosed herein may be separated by linker regions of various lengths. In some embodiments, the antigen binding domains are separated from each other, a CL, CH1, hinge, CH2, CH3, or the entire Fc region by a linker region. For example, the polypeptide chains may include the sequence VL1-CL-(linker) VH2-CH1, VH-linker-VL. Such linker regions may comprise a random assortment of amino acids or a restricted set of amino acids. Such linker regions can be flexible or rigid (see US2009/0155275). The inclusion of an amino acid linker has little or no influence on the activity of the antibodies disclosed herein.

Multi-specific antibodies have been constructed by genetically fusing two single chain Fv (scFv) or Fab fragments with or without the use of flexible linkers (Mallender et al., J. Biol. Chem. 1994 269:199-206; Mack et al., Proc. Natl. Acad. Sci. USA. 1995 92:7021-5; Zapata et al., Protein Eng. 1995 8.1057-62); via a dimerization device such as leucine zipper (Kostelny et al., J. Immunol. 1992148:1547-53; de Kruifetal J. Biol. Chem. 1996 271:7630-4) and Ig C/CH1 domains (Muller et al., FEBS Lett. 422:259-64); by diabody (Holliger et al., (1993) Proc. Nat. Acad. Sci. USA. 1998 90:6444-8; Zhu et al., Bio/Technology (NY) 1996 14:192-6); Fab-scFv fusion (Schoonjans et al., J. Immunol. 2000 165:7050-7); and mini antibody formats (Pack et al., Biochemistry 1992.31:1579-84; Pack et al., Bio/Technology 1993 11:1271-7).

The multi-specific antibodies and constructs as disclosed herein may include a linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of their antigen binding domains, CL domains, CH1 domains, hinge regions, CH2 domains, CH3 domains, or Fc regions. In some embodiments, the linker region is comprised of the amino acids glycine and serine. The linker may include the sequence GS (SEQ ID NO: 79), GGS (SEQ ID NO: 80), GSG (SEQ ID NO: 81), SGG (SEQ ID NO: 82), GGG (SEQ ID NO: 83), GGGS (SEQ ID NO: 84), SGGG (SEQ ID NO: 85), GGGGS (SEQ ID NO: 86), GGGGSGS (SEQ ID NO: 87), GGGGSGS (SEQ ID NO: 88), GGGGSGGS (SEQ ID NO: 89), GGGGSGGGGS (SEQ ID NO: 90), GGGGSGGGGSGGGGS (SEQ ID NO: 91), AKTTPKLEEGEFSEAR (SEQ ID NO: 92), AKTTPKLEEGEFSEARV (SEQ ID NO: 93), AKTTPKLGG (SEQ ID NO: 94), SAKTTPKLGG (SEQ ID NO: 95), AKTTPKLEEGEFSEARV (SEQ ID NO: 96), SAKTTP (SEQ ID NO: 97), SAKTTPKLGG (SEQ ID NO: 98), RADAAP (SEQ ID NO: 99), RADAAPTVS (SEQ ID NO: 100), RADAAAAGGPGS (SEQ ID NO: 101), RADAAAA(G4S)4 (SEQ ID NO: 102), SAKTTP (SEQ ID NO: 103), SAKTTPKLGG (SEQ ID NO: 104), SAKTTPKLEEGEFSEARV (SEQ ID NO: 105), ADAAP (SEQ ID NO: 106), ADAAPTVSIFPP (SEQ ID NO: 107), TVAAP (SEQ ID NO: 108), TVAAPSVFIFPP (SEQ ID NO: 109), QPKAAP (SEQ ID NO: 110), QPKAAPSVTLFPP (SEQ ID NO: 111), AKTTPP (SEQ ID NO: 112), AKTTPPSVTPLAP (SEQ ID NO: 113), AKTTAP (SEQ ID NO: 114), AKTTAPSVYPLAP (SEQ ID NO: 115), ASTKGP (SEQ ID NO: 116), ASTKGPSVFPLAP (SEQ ID NO: 117), GENKVEYAPALMALS (SEQ ID NO: 118), GPAKELTPLKEAKVS (SEQ ID NO: 119), and GHEAAAVMQVQYPAS (SEQ ID NO: 120), GGGGSGGGGSGGGGSGGGGS (SEQ ID NO: 121) or any combination thereof (see WO2007/024715).

### Dimerization-Specific Amino Acids

In one embodiment, the multivalent antibodies or constructs comprises at least one dimerization-specific amino acid change. The dimerization-specific amino acid change may result in “knob-into-hole” interactions, and may increase the likelihood of correct assembly of desired multivalent antibodies. The dimerization-specific amino acids may be within the CH1 domain or the CL domain or combinations thereof. Suitable dimerization-specific amino acids used to pair CH1 domains with other CH1 domains (CH1-CH1) and CL domains with other CL domains (CL-CL) may be found at least in the disclosures of WO2014082179, WO2015181805, and WO2017059551. The dimerization-specific amino acids can also be within the Fc domain and can be in combination with dimerization-specific amino acids within the CH1 or CL domains. In one embodiment, the present disclosure provides a bispecific antibody comprising at least one dimerization-specific amino acid pair.

### Antibody Production

Anti-B7H3 antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from Tables 1-2.

The polynucleotides of the present disclosure can encode the variable region sequence of an anti-B7H3 antibody. They can also encode both a variable region and a constant region of the antibody. Some of the polynucleotide sequences encode a polypeptide that comprises variable regions of both the heavy chain and the light chain of one of the exemplified anti-B7H3 antibodies.

In some embodiments, the polynucleotides described herein are codon-optimized for expression in host cells, e.g., eukaryotic cells, more specifically mammalian cells (e.g., CHO cells).

Also provided in the present disclosure are expression vectors and host cells for producing the anti-4Ig-B7H3 antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-4Ig-B7H3 antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-4Ig-B7H3 antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-4Ig-B7H3 antibody vectors can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-4Ig-B7H3 polypeptides. Insect cells in combination with baculovirus vectors can also be used.

In other aspects, mammalian host cells are used to express and produce the anti-4Ig-B7H3 polypeptides of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y, 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Methods of Detection and Diagnosis

The antibodies or antigen-binding fragments of the present disclosure are useful in a variety of applications including, but not limited to, methods for the detection of 4Ig-B7H3. In one aspect, the antibodies or antigen-binding fragments are useful for detecting the presence of 4Ig-B7H3 in a biological sample. The term “detecting” as used herein includes quantitative or qualitative detection. In certain aspects, a biological sample comprises a cell or tissue. In other aspects, such tissues include normal and/or cancerous tissues that express 4Ig-B7H3 at higher levels relative to other tissues.

In one aspect, the present disclosure provides a method of detecting the presence of 4Ig-B7H3 in a biological sample. In certain aspects, the method comprises contacting the biological sample with an anti-4Ig-B7H3 antibody or antigen binding fragment thereof under conditions permissive for binding of the antibody to the antigen and detecting whether a complex is formed between the antibody and the antigen. The biological sample can include, without limitation, urine, tissue, sputum, or blood.

Also included is a method of diagnosing a disorder associated with expression of 4Ig-B7H3. In certain aspects, the method comprises contacting a test cell with an anti-4Ig-B7H3 antibody; determining the level of expression (either quantitatively or qualitatively) of 4Ig-B7H3 expressed by the test cell by detecting binding of the anti-4Ig-B7H3 antibody to the 4Ig-B7H3 polypeptide; and comparing the level of expression by the test cell with the level of 4Ig-B7H3 expression in a control cell (e.g., a normal cell of the same tissue origin as the test cell or a non-4Ig-B7H3 expressing cell), wherein a higher level of 4Ig-B7H3 expression in the test cell as compared to the control cell indicates the presence of a disorder associated with expression of 4Ig-B7H3.

### Pharmaceutical Compositions and Formulations

Also provided are compositions, including pharmaceutical formulations, comprising an anti-4Ig-B7H3 antibody, antigen binding fragment thereof, multi-specific antibody, or polynucleotides comprising sequences encoding an anti-4Ig-B7H3 antibody, antigen binding fragment thereof, or multi-specific antibody. These compositions can further comprise suitable carriers, such as pharmaceutically acceptable excipients including buffers, which are well known in the art.

Pharmaceutical formulations of an anti-4Ig-B7H3 antibody or antigen binding fragment thereof as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility can be readily accomplished, e.g., by filtration through sterile filtration membranes.

### EQUIVALENTS

It is to be understood that, while the anti-human 4Ig-B7H3 antibodies and antigen binding fragments thereof have been described in conjunction with a detailed description thereof, the foregoing description is intended to illustrate and not limit the scope of the invention, which is defined by the scope of the appended claims. Other aspects, advantages, and modifications are within the scope of the claims below.

It is to be understood that one, some, any, or all of the features of the various embodiments disclosed herein may be combined to form further embodiments of the present disclosure. These and other aspects of the present disclosure will be apparent to those skilled in the art.

### EXAMPLES

### Example 1. Generation of Anti-huB7H3 VHH Antibody

### B7H3 Recombinant Proteins for Phage Campaign and Binding Assays

To discover VHH antibodies against human B7H3, several recombinant proteins were designed and expressed for phage panning and screening. The cDNA coding regions for the full-length human 4Ig-B7H3 (hu 4Ig-B7H3) were ordered based on the B7H3 GenBank sequence (Accession No: NM_001024736.1; the gene is available from Sinobio, Cat.: HG11188-M, and is referenced herein as SEQ ID NO: 1). In brief, the coding region of the extracellular domain (ECD) consisting of amino acid (AA) 29-466 of hu 4Ig-B7H3 (SEQ ID NO: 2) was PCR-amplified. The coding region of human IgG1 (huIgG1) Fc (SEQ ID NOs: 3-4) and mouse IgG2a (mIgG2a) Fc (SEQ ID NOs: 5-6) were PCR-amplified and then conjugated with ECD (SEQ ID NO: 58) of hu 4Ig-B7H3 by ligation to make huIgG1 Fc-fusion protein hu 4Ig-B7H3 ECD-huIgG1 and mIgG2a Fc-fusion protein hu 4Ig-B7H3 ECD-mIgG2a. PCR products were then cloned into a pcDNA3.1-based expression vector (Invitrogen, Carlsbad, CA, USA), which resulted in recombinant huIgG1 Fc-fusion protein and mIgG2a Fc-fusion protein expression plasmids. For recombinant fusion protein production, plasmids were transiently transfected into a HEK293-based mammalian cell expression system (developed in house) and cultured for 5-7 days in a CO₂ incubator equipped with a rotating shaker. The supernatants containing the recombinant proteins were collected and cleared by centrifugation. Recombinant proteins were purified by Protein A column (Cat.: 17127901, GE Life Sciences) or Ni-NTA agarose (Cat.: R90115, Invitrogen). All recombinant proteins were dialyzed against phosphate buffered saline (PBS) and stored in a −80° C. freezer in small aliquots.

### Llama Immunization and Phage Library Construction

One llama was immunized with hu 4Ig-B7H3 ECD-mIgG2a. Two weeks after the fourth immunization, llama PBMC were collected for RNA extraction, using standard techniques (P. Chomczynski, et al., Single-step method of RNA isolation by acid guanidinium thiocyanate-phenol-chloroform extraction, Analytical Biochem 162(1) 1987 156-159).

A phage library was constructed by reverse-transcription and splice-overlap extension PCR. The PCR products were double-digested by NcoI/NotI and ligated into the phagemid vector Pcantab-5E. Repertoires were then transformed into Escherichia coli TG1 bacteria and validated by DNA Sanger sequencing of random clones (>96 clones analyzed). Phages were purified by two precipitations with PEG/NaCl directly from the culture supernatant after a rescue step using KM13 helper phage. A library with size >10⁷ was obtained after transformation into E. coli bacteria.

### Phage Display Panning and Screening

Phage display selection was carried out by phage display using standard protocols (Silacci et al., (2005) Proteomics, 5, 2340-50; Zhao et al., (2014) PLoS One, 9, e111339). In brief, 10 μg/ml of immobilized hu 4Ig-B7H3 ECD-huIgG1 in immunotubes (Cat. 470319, ThermoFisher) was utilized in round 1 and 2. NK92mi cells overexpressing hu 4Ig-B7H3 (NK92mi/hu 4Ig-B7H3 cells) were used for selection in round 3. Immunotubes were blocked with 5% milk powder (w/v) in PBS for 1 hour. 2.69×10¹² (round 1) and 9.00×10¹² (round 2) phages were depleted by CD40-mIgG2a in PBS with 5% skim milk for 1 hour and then incubated with the antigen for 1 hour. For the third round of selection, cell panning was carried out using NK92mi/hu 4Ig-B7H3 cells with NK92mi cells as depletion cells. After washes with TBST, bound phages were eluted with 100 mM triethylamine (Sigma-Aldrich). Eluted phages were used to infect mid-log phase E. coli TG1 bacteria and plated onto 2X YT plates supplemented with 2% glucose and 100 μg/ml ampicillin. After three rounds of selections, individual clones were picked and phage-containing supernatants were prepared using standard protocols. Phage ELISA were used to screen anti-hu 4Ig-B7H3 VHH antibodies.

For phage ELISA, a Maxisorp immunoplate was coated with antigens and blocked with 3% milk powder (w/v) in PBS buffer. Phage supernatant was added to wells of the ELISA plate for 1 hour. After washes with TBST, bound phage was detected using HRP-conjugated anti-M13 antibody (SinoBiological) and 3,3′,5,5′-tetramethylbenzidine substrate (Invitrogen).

### Expression and Purification of Fc Fusion VHH Antibodies

Anti-hu 4Ig-B7H3 VHH antibodies were then constructed as human Fc fusion VHH antibody format (VHH-Fc) using in-house developed expression vectors. VHH domain antibodies were fused at the N terminal of human IgG1 Fc (SEQ ID NOs: 17-18). Expression and preparation of Fc fusion VHH antibodies were achieved by transfection into 293G cells and by purification using a Protein A column (GE Healthcare, 17-5438-02). The purified antibodies were concentrated in PBS and stored in aliquots in a −80° C. freezer.

### Example 2. Characterization of Purified Anti-Hu 4Ig-B7H3 VHH Antibodies

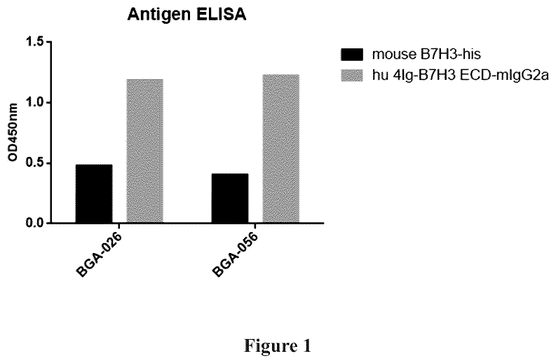

For antigen ELISA, a Maxisorp immunoplate was coated with antigen hu 4Ig-B7H3 ECD-mIgG2a or mouse B7H3-his (SinoBiological, 50973-M08H) and blocked with 3% BSA (w/v) in PBS buffer (blocking buffer). Monoclonal VHH antibodies were added to wells of the ELISA plate for 1 hour. After washes with TBST, bound antibodies were detected using HRP-conjugated anti-human IgG antibody (Sigma, A0170) and 3,3′,5,5′-tetramethylbenzidine substrate (Invitrogen). The ELISA analyses of two representative clones, BGA-026 and BGA-056, are shown in FIG. 1. The results show that BGA-026 (SEQ ID NOs: 7-11) and BGA-056 (SEQ ID NOs: 12-16) bind to hu 4Ig-B7H3 with good affinity compared with mouse B7H3-His (SinoBiological, 50973-M08H).

For affinity determination, the binding affinities between BGA-026 or BGA-056 and the antigen human B7H3 (ECD)-His (SinoBiological, 11188-H08H) were tested by surface plasmon resonance (SPR) technology. The results of SPR-determined binding profiles of BGA-026 and BGA-056 are summarized in Table 3. Both BGA-026 and BGA-056 showed good binding affinity with human B7H3.

TABLE 3
Binding affinities of BGA-026 and BGA-056 by SPR

| VHH | kₒₙ (M⁻¹s⁻¹) | k_{off} (s⁻¹) | K_{D} (M) |
|---|---|---|---|
| BGA-026 | 2.47E+05 | 2.78E−04 | 1.13E−09 |
| BGA-056 | 1.75E+05 | 1.07E−04 | 6.14E−10 |

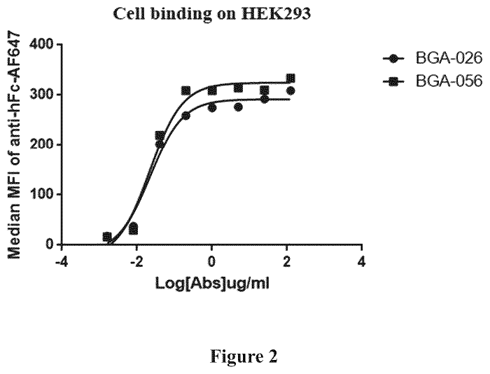

For cell binding analysis of BGA-026 and BGA-056, HEK293 cells were seeded in a 96-well plate, and were incubated with a diluted series of BGA-026 or BGA-056. Alexa Fluor® 647 anti-human IgG Fc Antibody (BioLegend, 410714) was used as a secondary antibody to detect antibody binding to the cell surface. EC50 values for dose-dependent binding to HEK293 cells were determined by fitting the dose-response data with GraphPad Prism. The results of cell binding profiles of BGA-026 and BGA-056 are summarized in FIG. 2 and Table 4.

TABLE 4
Binding of BGA-026 and BGA-056 to HEK293

| VHH | HEK293 binding EC50 (ug/ml) |
|---|---|
| BGA-026 | 0.02240 |
| BGA-056 | 0.02190 |

The amino acid and DNA sequences of BGA-026 and BGA-056 are shown in Table 5 below.

TABLE 5
Amino acid and DNA sequences of anti-B7H3 VHHs

| Antibody | SEQ ID NO | | Sequence |
|---|---|---|---|
| Human | SEQ ID NO: 1 | DNA | ATGCTGCGTCGGCGGGGCAGCCCTGGCATGGGTGTGC |
| B7H3-full | | | ATGTGGGTGCAGCCCTGGGAGCACTGTGGTTCTGCCT |
| length | | | CACAGGAGCCCTGGAGGTCCAGGTCCCTGAAGACCC |
| | | | AGTGGTGGCACTGGTGGGCACCGATGCCACCCTGTG |
| | | | CTGCTCCTTCTCCCCTGAGCCTGGCTTCAGCCTGGCA |
| | | | CAGCTCAACCTCATCTGGCAGCTGACAGATACCAAAC |
| | | | AGCTGGTGCACAGCTTTGCTGAGGGCCAGGACCAGG |
| | | | GCAGCGCCTATGCCAACCGCACGGCCCTCTTCCCGGA |
| | | | CCTGCTGGCACAGGGCAACGCATCCCTGAGGCTGCA |
| | | | GCGCGTGCGTGTGGCGGACGAGGGCAGCTTCACCTG |
| | | | CTTCGTGAGCATCCGGGATTTCGGCAGCGCTGCCGTC |
| | | | AGCCTGCAGGTGGCCGCTCCCTACTCGAAGCCCAGC |
| | | | ATGACCCTGGAGCCCAACAAGGACCTGCGGCCAGGG |
| | | | GACACGGTGACCATCACGTGCTCCAGCTACCAGGGCT |
| | | | ACCCTGAGGCTGAGGTGTTCTGGCAGGATGGGCAGG |
| | | | GTGTGCCCCTGACTGGCAACGTGACCACGTCGCAGAT |
| | | | GGCCAACGAGCAGGGCTTGTTTGATGTGCACAGCATC |
| | | | CTGCGGGTGGTGCTGGGTGCAAATGGCACCTACAGCT |
| | | | GCCTGGTGCGCAACCCCGTGCTGCAGCAGGATGCGC |
| | | | ACAGCTCTGTCACCATCACACCCCAGAGAAGCCCCA |
| | | | CAGGAGCCGTGGAGGTCCAGGTCCCTGAGGACCCGG |
| | | | TGGTGGCCCTAGTGGGCACCGATGCCACCCTGCGCTG |
| | | | CTCCTTCTCCCCCGAGCCTGGCTTCAGCCTGGCACAG |
| | | | CTCAACCTCATCTGGCAGCTGACAGACACCAAACAG |
| | | | CTGGTGCACAGTTTCACCGAAGGCCGGGACCAGGGC |
| | | | AGCGCCTATGCCAACCGCACGGCCCTCTTCCCGGACC |
| | | | TGCTGGCACAAGGCAATGCATCCCTGAGGCTGCAGC |
| | | | GCGTGCGTGTGGCGGACGAGGGCAGCTTCACCTGCT |
| | | | TCGTGAGCATCCGGGATTTCGGCAGCGCTGCCGTCAG |
| | | | CCTGCAGGTGGCCGCTCCCTACTCGAAGCCCAGCATG |
| | | | ACCCTGGAGCCCAACAAGGACCTGCGGCCAGGGGAC |
| | | | ACGGTGACCATCACGTGCTCCAGCTACCGGGGCTACC |
| | | | CTGAGGCTGAGGTGTTCTGGCAGGATGGGCAGGGTG |
| | | | TGCCCCTGACTGGCAACGTGACCACGTCGCAGATGG |
| | | | CCAACGAGCAGGGCTTGTTTGATGTGCACAGCGTCCT |
| | | | GCGGGTGGTGCTGGGTGCGAATGGCACCTACAGCTG |
| | | | CCTGGTGCGCAACCCCGTGCTGCAGCAGGATGCGCA |
| | | | CGGCTCTGTCACCATCACAGGGCAGCCTATGACATTC |
| | | | CCCCCAGAGGCCCTGTGGGTGACCGTGGGGCTGTCT |
| | | | GTCTGTCTCATTGCACTGCTGGTGGCCCTGGCTTTCGT |
| | | | GTGCTGGAGAAAGATCAAACAGAGCTGTGAGGAGGA |
| | | | GAATGCAGGAGCTGAGGACCAGGATGGGGAGGGAG |
| | | | AAGGCTCCAAGACAGCCCTGCAGCCTCTGAAACACT |
| | | | CTGACAGCAAAGAAGATGATGGACAAGAAATAGCCT |
| | | | GA |
| | SEQ ID NO: 2 | AA | MLRRRGSPGMGVHVGAALGALWFCLTGALEVQVPEDP |
| | | | VVALVGTDATLCCSFSPEPGFSLAQLNLIWQLTDTKQLV |
| | | | HSFAEGQDQGSAYANRTALFPDLLAQGNASLRLQRVRV |
| | | | ADEGSFTCFVSIRDFGSAAVSLQVAAPYSKPSMTLEPNK |
| | | | DLRPGDTVTITCSSYQGYPEAEVFWQDGQGVPLTGNVT |
| | | | TSQMANEQGLFDVHSILRVVLGANGTYSCLVRNPVLQQ |
| | | | DAHSSVTITPQRSPTGAVEVQVPEDPVVALVGTDATLRC |
| | | | SFSPEPGFSLAQLNLIWQLTDTKQLVHSFTEGRDQGSAY |
| | | | ANRTALFPDLLAQGNASLRLQRVRVADEGSFTCFVSIRD |
| | | | FGSAAVSLQVAAPYSKPSMTLEPNKDLRPGDTVTITCSS |
| | | | YRGYPEAEVFWQDGQGVPLTGNVTTSQMANEQGLFD |
| | | | VHSVLRVVLGANGTYSCLVRNPVLQQDAHGSVTITGQP |
| | | | MTFPPEALWVTVGLSVCLIALLVALAFVCWRKIKQSCE |
| | | | EENAGAEDQDGEGEGSKTALQPLKHSDSKEDDGQEIA |
| Hu 4Ig-B7H3 | SEQ ID NO: 58 | AA | LEVQVPEDPVVALVGTDATLCCSFSPEPGFSLAQLNLIW |
| ECD | | | QLTDTKQLVHSFAEGQDQGSAYANRTALFPDLLAQGNA |
| (AA 29-466 | | | SLRLQRVRVADEGSFTCFVSIRDFGSAAVSLQVAAPYSK |
| of hu 4Ig- | | | PSMTLEPNKDLRPGDTVTITCSSYQGYPEAEVFWQDGQ |
| B7H3 full | | | GVPLTGNVTTSQMANEQGLFDVHSILRVVLGANGTYSC |
| length) | | | LVRNPVLQQDAHSSVTITPQRSPTGAVEVQVPEDPVVAL |
| | | | VGTDATLRCSFSPEPGFSLAQLNLIWQLTDTKQLVHSFT |
| | | | EGRDQGSAYANRTALFPDLLAQGNASLRLQRVRVADEG |
| | | | SFTCFVSIRDFGSAAVSLQVAAPYSKPSMTLEPNKDLRP |
| | | | GDTVTITCSSYRGYPEAEVFWQDGQGVPLTGNVTTSQ |
| | | | MANEQGLFDVHSVLRVVLGANGTYSCLVRNPVLQQDA |
| | | | HGSVTITGQPMTFPPEA |
| huIgG1 Fc | SEQ ID NO: 3 | DNA | GCTAGCGAGCCCAAATCTTGTGACAAAACTCACACAT |
| | | | GCCCACCGTGCCCAGCACCTGAACTCCTGGGGGGAC |
| | | | CGTCAGTCTTCCTCTTCCCCCCAAAACCCAAGGACAC |
| | | | CCTCATGATCTCCCGGACCCCTGAGGTCACATGCGTG |
| | | | GTGGTGGACGTGAGCCACGAAGACCCTGAGGTCAAG |
| | | | TTCAACTGGTACGTGGACGGCGTGGAGGTGCATAATG |
| | | | CCAAGACAAAGCCGCGGGAGGAGCAGTACAACAGC |
| | | | ACGTACCGTGTGGTCAGCGTCCTCACCGTCCTGCACC |
| | | | AGGACTGGCTGAATGGCAAGGAGTACAAGTGCAAGG |
| | | | TCTCCAACAAAGCCCTCCCAGCCCCCATCGAGAAAA |
| | | | CCATCTCCAAAGCCAAAGGGCAGCCCCGAGAACCAC |
| | | | AGGTGTACACCCTGCCCCCATCCCGGGATGAGCTGAC |
| | | | CAAGAACCAGGTCAGCCTGACCTGCCTGGTCAAAGG |
| | | | CTTCTATCCCAGCGACATCGCCGTGGAGTGGGAGAGC |
| | | | AATGGGCAGCCGGAGAACAACTACAAGACCACGCCT |
| | | | CCCGTGCTGGACTCCGACGGCTCCTTCTTCCTCTACA |
| | | | GCAAGCTCACCGTGGACAAGAGCAGGTGGCAGCAGG |
| | | | GGAACGTCTTCTCATGCTCCGTGATGCATGAGGCTCT |
| | | | GCACAACCACTACACGCAGAAGAGCCTCTCCCTGTCT |
| | | | CCGGGTAAA |
| | SEQ ID NO: 4 | AA | ASEPKSCDKTHTCPPCPAPELLGGPSVFLFPPKPKDTLMI |
| | | | SRTPEVTCVVVDVSHEDPEVKFNWYVDGVEVHNAKTK |
| | | | PREEQYNSTYRVVSVLTVLHQDWLNGKEYKCKVSNKA |
| | | | LPAPIEKTISKAKGQPREPQVYTLPPSRDELTKNQVSLTC |
| | | | LVKGFYPSDIAVEWESNGQPENNYKTTPPVLDSDGSFFL |
| | | | YSKLTVDKSRWQQGNVFSCSVMHEALHNHYTQKSLSL |
| | | | SPGK |
| mIgG2a Fc | SEQ ID NO: 5 | DNA | GCTAGCGAGCCCAGAGGGCCCACAATCAAGCCCTGT |
| | | | CCTCCATGCAAATGCCCAGCACCTAACCTCTTGGGTG |
| | | | GACCATCCGTCTTCATCTTCCCTCCAAAGATCAAGGAT |
| | | | GTACTCATGATCTCCCTGAGCCCCATGGTCACATGTGT |
| | | | GGTGGTGGATGTGAGCGAGGATGACCCAGATGTCCA |
| | | | GATCAGCTGGTTCGTGAACAACGTGGAAGTACTCACA |
| | | | GCTCAGACACAAACCCATAGAGAGGATTACAACAGT |
| | | | ACTCTCCGGGTGGTCAGTGCCCTCCCCATCCAGCACC |
| | | | AGGACTGGATGAGTGGCAAGGAGTTCAAATGCAAGG |
| | | | TCAACAACAAAGCCCTCCCAGCGCCCATCGAGAGAA |
| | | | CCATCTCAAAACCCAAAGGGTCAGTAAGAGCTCCAC |
| | | | AGGTATATGTCTTGCCTCCACCAGAAGAAGAGATGAC |
| | | | TAAGAAACAGGTCACTCTGACCTGCATGGTCACAGAC |
| | | | TTCATGCCTGAAGACATTTACGTGGAGTGGACCAACA |
| | | | ACGGGAAAACAGAGCTAAACTACAAGAACACTGAAC |
| | | | CAGTCCTGGACTCTGATGGTTCTTACTTCATGTACAGC |
| | | | AAGCTGAGAGTGGAAAAGAAGAACTGGGTGGAAAG |
| | | | AAATAGCTACTCCTGCTCAGTGGTCCACGAGGGTCTG |
| | | | CACAATCACCACACGACTAAGAGCTTCTCCCGGACTC |
| | | | CGGGTAAA |
| | SEQ ID NO: 6 | AA | ASEPRGPTIKPCPPCKCPAPNLLGGPSVFIFPPKIKDVLMI |
| | | | SLSPMVTCVVVDVSEDDPDVQISWFVNNVEVLTAQTQT |
| | | | HREDYNSTLRVVSALPIQHQDWMSGKEFKCKVNNKAL |
| | | | PAPIERTISKPKGSVRAPQVYVLPPPEEEMTKKQVTLTC |
| | | | MVTDFMPEDIYVEWTNNGKTELNYKNTEPVLDSDGSY |
| | | | FMYSKLRVEKKNWVERNSYSCSVVHEGLHNHHTTKSF |
| | | | SRTPGK |
| BGA-026 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 10 | VHH AA | EVQLVESGGGLVQTGDSLRLSCAASPRTFQTYFMGWFR |
| | | | QAPGKEREFVSVINWSGGTSYYKDSVKGRFTISRDNAK |
| | | | NTVYLQMNNLKPEDTAVYYCAAKGSGTYDREGEYDY |
| | | | WGQGTQVTVSS |
| | SEQ ID NO: 11 | VHH DNA | GAGGTGCAGCTGGTGGAGTCTGGGGGAGGATTGGTG |
| | | | CAGACTGGGGACTCTCTGAGACTCTCCTGTGCAGCCT |
| | | | CTCCACGCACCTTCCAGACCTATTTCATGGGCTGGTTC |
| | | | CGCCAGGCTCCAGGGAAGGAGCGTGAGTTTGTATCA |
| | | | GTTATTAACTGGAGTGGCGGTACCTCATACTATAAAGA |
| | | | TTCCGTGAAGGGCCGATTCACCATCTCCAGAGACAAC |
| | | | GCCAAGAATACCGTGTATCTACAAATGAACAACCTGA |
| | | | AACCTGAGGACACGGCCGTTTATTACTGTGCAGCTAA |
| | | | GGGGTCCGGTACTTACGACCGAGAGGGGGAATATGAC |
| | | | TACTGGGGCCAGGGGACCCAGGTCACCGTCTCCTCA |
| BGA-056 | SEQ ID NO: 12 | CDR1 | DYHMS |
| | | (Kabat) | |
| | SEQ ID NO: 13 | CDR2 | GILNTGLSPTYAATVTG |
| | | (Kabat) | |
| | SEQ ID NO: 14 | CDR3 | GYYRATPSQAN |
| | | (Kabat) | |
| | SEQ ID NO: 15 | VHH AA | EVQLLESGGGLVQPGGSLRLSCAASGFTFSDYHMSWVR |
| | | | QAPGKGLEWVSGILNTGLSPTYAATVTGRFTISRDNAK |
| | | | NTLYLSMNSLKPEDTAVYYCAQGYYRATPSQANRGQG |
| | | | TQVTVSS |
| | SEQ ID NO: 16 | VHH DNA | GAGGTGCAGCTGCTGGAGTCTGGGGGAGGCTTGGTG |
| | | | CAGCCTGGGGGGTCTCTGAGACTCTCCTGTGCAGCCT |
| | | | CTGGATTCACCTTCAGTGATTATCACATGAGCTGGGT |
| | | | CCGCCAGGCTCCAGGAAAGGGGCTCGAGTGGGTCTC |
| | | | AGGTATTCTTAATACAGGTCTTAGTCCAACTTATGCA |
| | | | GCCACCGTGACGGGCCGATTCACCATCTCCAGAGAC |
| | | | AACGCCAAGAATACGCTGTATCTCTCAATGAACAGC |
| | | | CTGAAACCTGAGGACACGGCCGTGTATTATTGTGCCC |
| | | | AGGGATACTATCGAGCTACCCCCTCCCAAGCCAACA |
| | | | GGGGCCAGGGGACCCAGGTCACCGTCTCCTCA |
| human IgG1 | SEQ ID NO: 17 | DNA | GAACCTAAATCATCAGATAAGACTCATACATGCCCTCC |
| Fc | | | TTGCCCTGCACCTGAACTTCTTGGAGGACCTTCAGTG |
| | | | TTCCTATTTCCACCGAAACCTAAAGATACACTTATGAT |
| | | | CTCAAGAACACCTGAAGTGACATGCGTGGTGGTGGA |
| | | | TGTGTCACACGAAGATCCTGAAGTGAAATTTAACTGG |
| | | | TACGTGGATGGAGTGGAAGTGCACAACGCAAAGACC |
| | | | AAGCCTAGAGAAGAACAATACAACTCAACATACAGA |
| | | | GTGGTGTCAGTGCTTACAGTGCTTCACCAAGATTGGC |
| | | | TTAACGGAAAGGAGTATAAATGCAAAGTGTCAAACA |
| | | | AAGCACTTCCTGCACCTATCGAGAAGACCATTTCAAA |
| | | | AGCAAAAGGACAACCTAGAGAACCTCAAGTGTACAC |
| | | | ACTTCCTCCTTCAAGAGAAGAAATGACAAAGAATCA |
| | | | GGTGTCGCTGACTTGTCTCGTCAAAGGATTCTATCCAT |
| | | | CAGATATCGCAGTGGAATGGGAATCAAACGGACAACC |
| | | | TGAGAATAATTACAAGACTACGCCTCCTGTGCTTGATT |
| | | | CAGATGGATCATTCTTCCTATATTCAAAGCTGACGGTG |
| | | | GATAAATCAAGATGGCAACAAGGAAACGTGTTCTCCT |
| | | | GTTCAGTGATGCACGAAGCACTTCACAACCACTACAC |
| | | | ACAGAAGTCCTTATCGTTGTCTCCCGGAAAG |
| | SEQ ID NO: 18 | AA | EPKSSDKTHTCPPCPAPELLGGPSVFLFPPKPKDTLMISR |
| | | | TPEVTCVVVDVSHEDPEVKFNWYVDGVEVHNAKTKP |
| | | | REEQYNSTYRVVSVLTVLHQDWLNGKEYKCKVSNKAL |
| | | | PAPIEKTISKAKGQPREPQVYTLPPSREEMTKNQVSLTC |
| | | | LVKGFYPSDIAVEWESNGQPENNYKTTPPVLDSDGSFFL |
| | | | YSKLTVDKSRWQQGNVFSCSVMHEALHNHYTQKSLSL |
| | | | SPGK |

### Example 3. Humanization of BGA-026

For humanization of BGA-026, human germline IgG genes were searched for sequences that share high degrees of homology to the protein sequences of BGA-026 variable regions by blasting against the human immunoglobulin gene database in IMGT. The human IGVH 3-23*04 gene that is present in human antibody repertoires with high frequency and high homology to BGA-026 was selected as the template for humanization.

The humanized variants were constructed in a VHH-human IgG1 Fc (SEQ ID NO: 18) format using an in-house developed expression vector, with easy adapting sub-cloning sites. The humanized variants were expressed by transfection of the constructs into Expi293F cells and purified using a protein A column (Cat: 17-5438-02, GE Life Sciences). The purified antibody was concentrated to 0.5-5 mg/ml in PBS and stored in aliquots in a −80° C. freezer.

### Framework Swapping Based on BGA-026

Humanization was carried out by CDR-grafting with key back mutations retained. The humanized VHH antibodies from BGA-026 were engineered in a VHH-human IgG1 Fc format using an in-house developed expression vector. In the initial round of humanization, mutations from camelid variable region to human amino acid residues in framework regions were guided by 3D structure analysis. The camelid framework residues with structural importance for maintaining the canonical structural of CDRs were retained in the first round of humanization, including nine amino acid residues—P26, R27, Q30, F37, E44, R45, F47, V78, and A94 (Kabat numbering)—resulting in BGA-154 (Table 9; SEQ ID NOs: 20 to 21). The binding affinity between BGA-154 and human B7H3 (ECD)-His (Cat: 11188-H08H, SinoBiological) was tested using SPR. BGA-154 has good binding affinity, comparable with the chimeric camelid antibody BGA-026 (Table 6).

Variants with Point Mutations Based on BGA-154

Single point mutation based on BGA-154 was performed for the above nine back mutations to explore their significance (BGA-1541 to BGA-1549; Table 9; SEQ ID NOs: 22 to 39).

Binding affinities of BGA-1541 to BGA-1549 were tested by SPR using human B7H3 (ECD)-His (Cat: 11188-H08H, SinoBiological) and are shown in Table 6. SPR analysis of BGA-1541 to BGA-1549 determined three critical back mutation sites: P26, F37, and A94.

TABLE 6
Binding affinities of variants with point mutations based on BGA-154

| | | | | | K_{D} ratio to | SEC- |
|---|---|---|---|---|---|---|
| | | | | | BGA-026 | HPLC |
| VHH | Mutations | kₒₙ (M⁻¹s⁻¹) | k_{off} (s⁻¹) | K_{D} (M) | chimeric | purity |
| BGA-026 | | 9.74E+05 | 2.55E−04 | 2.62E−10 | 1.00 | 97.74 |
| BGA-154 | P26, R27, | 8.53E+05 | 2.01E−04 | 2.36E−10 | 0.90 | 96.01 |
| | Q30, F37, | | | | | |
| | E44, R45, | | | | | |
| | F47, V78, | | | | | |
| | A94 | | | | | |
| BGA-1541 | P26G | No binding | No binding | No binding | — | — |
| BGA-1542 | R27F | 1.24E+06 | 5.39E−04 | 4.34E−10 | 1.66 | 94.06 |
| BGA-1543 | Q30S | 1.23E+06 | 6.16E−04 | 5.01E−10 | 1.91 | 96.74 |
| BGA-1544 | F37V | No | No | No | — | — |
| | | expression | expression | expression | | |
| BGA-1545 | E44G | 8.98E+05 | 2.36E−04 | 2.63E−10 | 1.00 | 96.98 |
| BGA-1546 | R45L | 9.24E+05 | 2.73E−04 | 2.95E−10 | 1.13 | 97.18 |
| BGA-1547 | F47W | 1.65E+06 | 4.38E−04 | 2.65E−10 | 1.01 | 95.78 |
| BGA-1548 | V78L | 9.01E+05 | 2.05E−04 | 2.27E−10 | 0.87 | 95.61 |
| BGA-1549 | A94K | No | No | No | — | — |
| | | expression | expression | expression | | |

Keeping the three amino acids (P26, F37, A94) back mutated, combinational mutation of the other six amino acid sites was carried out to generate BGA-2041 to BGA-2046 (Table 7; SEQ ID NOs: 40 to 51). Binding affinities of BGA-2041 to BGA-2046 tested by SPR are shown in Table 7. BGA-2041 to BGA-2046 all have good binding affinity to human B7H3. SPR analysis of BGA-2041 to BGA-2046 revealed that variants BGA-2041, BGA-2042, and BGA-2046 have comparable affinity to that of BGA-026 chimeric antibody.

Anti-hu 4Ig-B7H3 VHH antibodies were constructed as human Fc fusion VHH antibody format (VHH-Fc) using in-house developed expression vectors. Expression and preparation of Fc fusion VHH antibodies were achieved by transfection into 293G cells and by purification using a Protein A column (GE Healthcare, 17-5438-02). The purified antibodies were concentrated in PBS and stored in aliquots in a −80° C. freezer. All of the antibodies tested (see Table 6 and Table 7) except BGA-1541, BGA-1544, and BGA-1549, exhibit good affinity and high purity, and are easy to purify.

TABLE 7
Binding affinities of variants with combinational
mutations based on BGA-154

| | | | | K_{D} ratio | |
|---|---|---|---|---|---|
| | | | | to | SEC- |
| | | | | BGA-026 | HPLC |
| VHH | kₒₙ (M⁻¹s⁻¹) | k_{off} (s⁻¹) | K_{D} (M) | chimeric | purity |
| BGA-026 | 7.04E+05 | 3.42E−04 | 4.86E−10 | 1.00 | 97.74 |
| BGA-2041 | 4.11E+05 | 3.88E−04 | 9.44E−10 | 1.94 | 94.37 |
| BGA-2042 | 4.48E+05 | 3.70E−04 | 8.25E−10 | 1.70 | 97.87 |
| BGA-2043 | 4.15E+05 | 1.05E−03 | 2.53E−09 | 5.21 | 97.54 |
| BGA-2044 | 5.37E+05 | 4.96E−03 | 9.25E−09 | 19.03 | 96.97 |
| BGA-2045 | 6.46E+05 | 1.46E−02 | 2.25E−08 | 46.30 | 97.49 |
| BGA-2046 | 4.36E+05 | 2.67E−04 | 6.12E−10 | 1.26 | 97.68 |

Cell Binding Determination of Humanized Variants from BGA-026

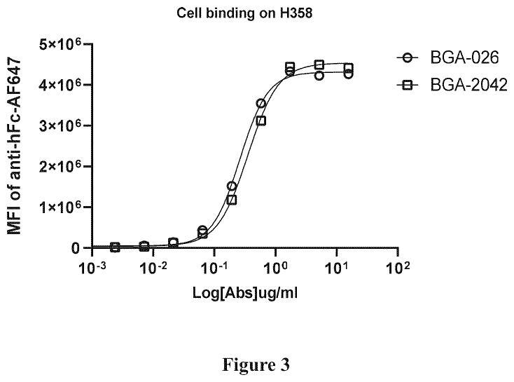

Non-small cell lung cancer cell line H358, which natively expresses high levels of B7H3, was used to evaluate the binding activity of BGA-026 chimeric antibody and humanized variant BGA-2042. H358 cells were seeded in a 96-well plate, and were incubated with a diluted series of BGA-026 chimeric antibody and humanized variant BGA-2042. Goat anti-human IgG was used as a secondary antibody to detect antibody binding to the cell surface. EC50 values for dose-dependent binding to H358 cells were determined by fitting the dose-response data to the four-parameter logistic model with GraphPad Prism. The data shown in FIG. 3 and Table 8 demonstrate that the humanized variant BGA-2042 exhibits comparable binding affinity to BGA-026 chimeric antibody and has the ability to bind to native B7H3 on cells.

TABLE 8
Binding of chimeric BGA-026 and humanized
VHH BGA-2042 to H358 cells

| VHH | H358 binding EC50 (ug/mL) |
|---|---|
| chimeric BGA-026 | 0.263 |
| BGA-2042 | 0.357 |

TABLE 9
Amino acid and DNA sequences of BGA-026 and the humanized variants thereof

| Antibody | SEQ ID NO | | SEQUENCE |
|---|---|---|---|
| BGA-026 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 10 | VHH AA | EVQLVESGGGLVQTGDSLRLSCAASPRTFQTYFMGW |
| | | | FRQAPGKEREFVSVINWSGGTSYYKDSVKGRFTISRD |
| | | | NAKNTVYLQMNNLKPEDTAVYYCAAKGSGTYDREG |
| | | | EYDYWGQGTQVTVSS |
| | SEQ ID NO: 19 | VHH DNA | GAAGTGCAACTGGTGGAGTCCGGCGGCGGGCTGG |
| | | | TCCAAACCGGGGACAGCCTGAGACTGAGCTGCGC |
| | | | CGCCTCCCCTAGAACCTTTCAGACCTACTTCATGGG |
| | | | CTGGTTCAGACAAGCCCCCGGCAAGGAGAGAGAG |
| | | | TTCGTGAGCGTGATCAACTGGAGCGGCGGCACAAG |
| | | | CTACTACAAGGACAGCGTGAAGGGCAGATTCACCA |
| | | | TCAGCAGAGACAACGCCAAGAACACAGTGTACCT |
| | | | GCAGATGAACAACCTGAAGCCCGAGGACACCGCC |
| | | | GTGTACTACTGCGCCGCCAAGGGCAGCGGCACCTA |
| | | | CGACAGAGAGGGCGAGTACGACTACTGGGGCCAA |
| | | | GGCACCCAAGTGACCGTGAGCAGC |
| BGA-154 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 20 | VHH AA | EVQLVESGGGLVQPGGSLRLSCAASPRTFQTYFMGW |
| | | | FRQAPGKEREFVSVINWSGGTSYYKDSVKGRFTISRD |
| | | | NSKNTVYLQMNSLRAEDTAVYYCAAKGSGTYDREG |
| | | | EYDYWGQGTLVTVSS |
| | SEQ ID NO: 21 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTCAGACCTACTTCATGGG |
| | | | CTGGTTCAGACAAGCCCCCGGCAAGGAGAGAGAG |
| | | | TTCGTGAGCGTGATCAACTGGAGCGGCGGCACAA |
| | | | GCTACTACAAGGACAGCGTGAAGGGCAGATTCAC |
| | | | CATCAGCAGAGACAACAGCAAGAACACCGTGTAC |
| | | | CTGCAGATGAACAGCCTGAGAGCCGAGGACACCG |
| | | | CCGTGTACTACTGCGCCGCCAAGGGCAGCGGCACC |
| | | | TACGACAGAGAGGGCGAGTACGACTACTGGGGCC |
| | | | AAGGCACCCTGGTGACCGTGAGCAGC |
| BGA-1541 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 22 | VHH AA | EVQLVESGGGLVQPGGSLRLSCAASGRTFQTYFMG |
| | | | WFRQAPGKEREFVSVINWSGGTSYYKDSVKGRFTIS |
| | | | RDNSKNTVYLQMNSLRAEDTAVYYCAAKGSGTYDR |
| | | | EGEYDYWGQGTLVTVSS |
| | SEQ ID NO: 23 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCGGCAGAACCTTTCAGACCTACTTCATGGG |
| | | | CTGGTTCAGACAAGCCCCCGGCAAGGAGAGAGAG |
| | | | TTCGTGAGCGTGATCAACTGGAGCGGCGGCACAA |
| | | | GCTACTACAAGGACAGCGTGAAGGGCAGATTCAC |
| | | | CATCAGCAGAGACAACAGCAAGAACACCGTGTAC |
| | | | CTGCAGATGAACAGCCTGAGAGCCGAGGACACCG |
| | | | CCGTGTACTACTGCGCCGCCAAGGGCAGCGGCACC |
| | | | TACGACAGAGAGGGCGAGTACGACTACTGGGGCC |
| | | | AAGGCACCCTGGTGACCGTGAGCAGC |
| BGA-1542 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 24 | VHH AA | EVQLVESGGGLVQPGGSLRLSCAASPFTFQTYFMGW |
| | | | FRQAPGKEREFVSVINWSGGTSYYKDSVKGRFTISRD |
| | | | NSKNTVYLQMNSLRAEDTAVYYCAAKGSGTYDREG |
| | | | EYDYWGQGTLVTVSS |
| | SEQ ID NO: 25 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTTTCACCTTTCAGACCTACTTCATGGGC |
| | | | TGGTTCAGACAAGCCCCCGGCAAGGAGAGAGAGT |
| | | | TCGTGAGCGTGATCAACTGGAGCGGCGGCACAAGC |
| | | | TACTACAAGGACAGCGTGAAGGGCAGATTCACCAT |
| | | | CAGCAGAGACAACAGCAAGAACACCGTGTACCTG |
| | | | CAGATGAACAGCCTGAGAGCCGAGGACACCGCCG |
| | | | TGTACTACTGCGCCGCCAAGGGCAGCGGCACCTAC |
| | | | GACAGAGAGGGCGAGTACGACTACTGGGGCCAAG |
| | | | GCACCCTGGTGACCGTGAGCAGC |
| BGA-1543 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 26 | VHH AA | EVQLVESGGGLVQPGGSLRLSCAASPRTFSTYFMGW |
| | | | FRQAPGKEREFVSVINWSGGTSYYKDSVKGRFTISRD |
| | | | NSKNTVYLQMNSLRAEDTAVYYCAAKGSGTYDREG |
| | | | EYDYWGQGTLVTVSS |
| | SEQ ID NO: 27 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTAGCACCTACTTCATGGGC |
| | | | TGGTTCAGACAAGCCCCCGGCAAGGAGAGAGAGT |
| | | | TCGTGAGCGTGATCAACTGGAGCGGCGGCACAAGC |
| | | | TACTACAAGGACAGCGTGAAGGGCAGATTCACCAT |
| | | | CAGCAGAGACAACAGCAAGAACACCGTGTACCTG |
| | | | CAGATGAACAGCCTGAGAGCCGAGGACACCGCCG |
| | | | TGTACTACTGCGCCGCCAAGGGCAGCGGCACCTAC |
| | | | GACAGAGAGGGCGAGTACGACTACTGGGGCCAAG |
| | | | GCACCCTGGTGACCGTGAGCAGC |
| BGA-1544 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 28 | VHH AA | EVQLVESGGGLVQPGGSLRLSCAASPRTFQTYFMGW |
| | | | VRQAPGKEREFVSVINWSGGTSYYKDSVKGRFTISR |
| | | | DNSKNTVYLQMNSLRAEDTAVYYCAAKGSGTYDRE |
| | | | GEYDYWGQGTLVTVSS |
| | SEQ ID NO: 29 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTCAGACCTACTTCATGGGC |
| | | | TGGGTGAGACAAGCCCCCGGCAAGGAGAGAGAGT |
| | | | TCGTGAGCGTGATCAACTGGAGCGGCGGCACAAGC |
| | | | TACTACAAGGACAGCGTGAAGGGCAGATTCACCAT |
| | | | CAGCAGAGACAACAGCAAGAACACCGTGTACCTG |
| | | | CAGATGAACAGCCTGAGAGCCGAGGACACCGCCG |
| | | | TGTACTACTGCGCCGCCAAGGGCAGCGGCACCTAC |
| | | | GACAGAGAGGGCGAGTACGACTACTGGGGCCAAG |
| | | | GCACCCTGGTGACCGTGAGCAGC |
| BGA-1545 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 30 | VHH AA | EVQLVESGGGLVQPGGSLRLSCAASPRTFQTYFMGW |
| | | | FRQAPGKGREFVSVINWSGGTSYYKDSVKGRFTISRD |
| | | | NSKNTVYLQMNSLRAEDTAVYYCAAKGSGTYDREG |
| | | | EYDYWGQGTLVTVSS |
| | SEQ ID NO: 31 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTCAGACCTACTTCATGGGC |
| | | | TGGTTCAGACAAGCCCCCGGCAAGGGCAGAGAGT |
| | | | TCGTGAGCGTGATCAACTGGAGCGGCGGCACAAGC |
| | | | TACTACAAGGACAGCGTGAAGGGCAGATTCACCAT |
| | | | CAGCAGAGACAACAGCAAGAACACCGTGTACCTG |
| | | | CAGATGAACAGCCTGAGAGCCGAGGACACCGCCG |
| | | | TGTACTACTGCGCCGCCAAGGGCAGCGGCACCTAC |
| | | | GACAGAGAGGGCGAGTACGACTACTGGGGCCAAG |
| | | | GCACCCTGGTGACCGTGAGCAGC |
| BGA-1546 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 32 | VHH AA | EVQLVESGGGLVQPGGSLRLSCAASPRTFQTYFMGW |
| | | | FRQAPGKELEFVSVINWSGGTSYYKDSVKGRFTISRD |
| | | | NSKNTVYLQMNSLRAEDTAVYYCAAKGSGTYDREG |
| | | | EYDYWGQGTLVTVSS |
| | SEQ ID NO: 33 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTCAGACCTACTTCATGGGC |
| | | | TGGTTCAGACAAGCCCCCGGCAAGGAGCTGGAGTT |
| | | | CGTGAGCGTGATCAACTGGAGCGGCGGCACAAGCT |
| | | | ACTACAAGGACAGCGTGAAGGGCAGATTCACCATC |
| | | | AGCAGAGACAACAGCAAGAACACCGTGTACCTGC |
| | | | AGATGAACAGCCTGAGAGCCGAGGACACCGCCGT |
| | | | GTACTACTGCGCCGCCAAGGGCAGCGGCACCTACG |
| | | | ACAGAGAGGGCGAGTACGACTACTGGGGCCAAGG |
| | | | CACCCTGGTGACCGTGAGCAGC |
| BGA-1547 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 34 | VHH AA | EVQLVESGGGLVQPGGSLRLSCAASPRTFQTYFMGW |
| | | | FRQAPGKEREWVSVINWSGGTSYYKDSVKGRFTISR |
| | | | DNSKNTVYLQMNSLRAEDTAVYYCAAKGSGTYDRE |
| | | | GEYDYWGQGTLVTVSS |
| | SEQ ID NO: 35 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTCAGACCTACTTCATGGGC |
| | | | TGGTTCAGACAAGCCCCCGGCAAGGAGAGAGAGT |
| | | | GGGTGAGCGTGATCAACTGGAGCGGCGGCACAAG |
| | | | CTACTACAAGGACAGCGTGAAGGGCAGATTCACCA |
| | | | TCAGCAGAGACAACAGCAAGAACACCGTGTACCT |
| | | | GCAGATGAACAGCCTGAGAGCCGAGGACACCGCC |
| | | | GTGTACTACTGCGCCGCCAAGGGCAGCGGCACCTA |
| | | | CGACAGAGAGGGCGAGTACGACTACTGGGGCCAA |
| | | | GGCACCCTGGTGACCGTGAGCAGC |
| BGA-1548 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 36 | VHH AA | EVQLVESGGGLVQPGGSLRLSCAASPRTFQTYFMGW |
| | | | FRQAPGKEREFVSVINWSGGTSYYKDSVKGRFTISRD |
| | | | NSKNTLYLQMNSLRAEDTAVYYCAAKGSGTYDREG |
| | | | EYDYWGQGTLVTVSS |
| | SEQ ID NO: 37 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTCAGACCTACTTCATGGGC |
| | | | TGGTTCAGACAAGCCCCCGGCAAGGAGAGAGAGT |
| | | | TCGTGAGCGTGATCAACTGGAGCGGCGGCACAAGC |
| | | | TACTACAAGGACAGCGTGAAGGGCAGATTCACCAT |
| | | | CAGCAGAGACAACAGCAAGAACACCCTGTACCTG |
| | | | CAGATGAACAGCCTGAGAGCCGAGGACACCGCCG |
| | | | TGTACTACTGCGCCGCCAAGGGCAGCGGCACCTAC |
| | | | GACAGAGAGGGCGAGTACGACTACTGGGGCCAAG |
| | | | GCACCCTGGTGACCGTGAGCAGC |
| BGA-1549 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 38 | VHH AA | EVQLVESGGGLVQPGGSLRLSCAASPRTFQTYFMGW |
| | | | FRQAPGKEREFVSVINWSGGTSYYKDSVKGRFTISRD |
| | | | NSKNTVYLQMNSLRAEDTAVYYCAKKGSGTYDREG |
| | | | EYDYWGQGTLVTVSS |
| | SEQ ID NO: 39 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTCAGACCTACTTCATGGGC |
| | | | TGGTTCAGACAAGCCCCCGGCAAGGAGAGAGAGT |
| | | | TCGTGAGCGTGATCAACTGGAGCGGCGGCACAAGC |
| | | | TACTACAAGGACAGCGTGAAGGGCAGATTCACCAT |
| | | | CAGCAGAGACAACAGCAAGAACACCGTGTACCTG |
| | | | CAGATGAACAGCCTGAGAGCCGAGGACACCGCCG |
| | | | TGTACTACTGCGCCAAGAAGGGCAGCGGCACCTAC |
| | | | GACAGAGAGGGCGAGTACGACTACTGGGGCCAAG |
| | | | GCACCCTGGTGACCGTGAGCAGC |
| BGA-2041 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 40 | VHH AA | EVQLVESGGGLVQPGGSLRLSCAASPRTFQTYFMG |
| | | | WFRQAPGKGLEWVSVINWSGGTSYYKDSVKGRF |
| | | | TISRDNSKNTLYLQMNSLRAEDTAVYYCAAKGSG |
| | | | TYDREGEYDYWGQGTLVTVSS |
| | SEQ ID NO: 41 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTCAGACCTACTTCATGGGC |
| | | | TGGTTCAGACAAGCCCCCGGCAAGGGCCTGGAGT |
| | | | GGGTGAGCGTGATCAACTGGAGCGGCGGCACAAG |
| | | | CTACTACAAGGACAGCGTGAAGGGCAGATTCACCA |
| | | | TCAGCAGAGACAACAGCAAGAACACCCTGTACCT |
| | | | GCAGATGAACAGCCTGAGAGCCGAGGACACCGCC |
| | | | GTGTACTACTGCGCCGCCAAGGGCAGCGGCACCTA |
| | | | CGACAGAGAGGGCGAGTACGACTACTGGGGCCAA |
| | | | GGCACCCTGGTGACCGTGAGCAGC |
| BGA-2042 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 42 | VHH AA | EVQLVESGGGLVQPGGSLRLSCAASPRTFQTYFMG |
| | | | WFRQAPGKELEWVSVINWSGGTSYYKDSVKGRF |
| | | | TISRDNSKNTLYLQMNSLRAEDTAVYYCAAKGSG |
| | | | TYDREGEYDYWGQGTLVTVSS |
| | SEQ ID NO: 43 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTCAGACCTACTTCATGGGC |
| | | | TGGTTCAGACAAGCCCCCGGCAAGGAGCTGGAGT |
| | | | GGGTGAGCGTGATCAACTGGAGCGGCGGCACAAG |
| | | | CTACTACAAGGACAGCGTGAAGGGCAGATTCACCA |
| | | | TCAGCAGAGACAACAGCAAGAACACCCTGTACCT |
| | | | GCAGATGAACAGCCTGAGAGCCGAGGACACCGCC |
| | | | GTGTACTACTGCGCCGCCAAGGGCAGCGGCACCTA |
| | | | CGACAGAGAGGGCGAGTACGACTACTGGGGCCAA |
| | | | GGCACCCTGGTGACCGTGAGCAGC |
| BGA-2043 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 44 | VHH AA | EVQLVESGGGLVQPGGSLRLSCAASPFTFQTYFMG |
| | | | WFRQAPGKELEWVSVINWSGGTSYYKDSVKGRF |
| | | | TISRDNSKNTLYLQMNSLRAEDTAVYYCAAKGSG |
| | | | TYDREGEYDYWGQGTLVTVSS |
| | SEQ ID NO: 45 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTTTCACCTTTCAGACCTACTTCATGGGC |
| | | | TGGTTCAGACAAGCCCCCGGCAAGGAGCTGGAGT |
| | | | GGGTGAGCGTGATCAACTGGAGCGGCGGCACAAG |
| | | | CTACTACAAGGACAGCGTGAAGGGCAGATTCACCA |
| | | | TCAGCAGAGACAACAGCAAGAACACCCTGTACCT |
| | | | GCAGATGAACAGCCTGAGAGCCGAGGACACCGCC |
| | | | GTGTACTACTGCGCCGCCAAGGGCAGCGGCACCTA |
| | | | CGACAGAGAGGGCGAGTACGACTACTGGGGCCAA |
| | | | GGCACCCTGGTGACCGTGAGCAGC |
| BGA-2044 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 46 | VHH AA | EVQLVESGGGLVQPGGSLRLSCAASPRTFSTYFMG |
| | | | WFRQAPGKELEWVSVINWSGGTSYYKDSVKGRF |
| | | | TISRDNSKNTLYLQMNSLRAEDTAVYYCAAKGSG |
| | | | TYDREGEYDYWGQGTLVTVSS |
| | SEQ ID NO: 47 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTAGCACCTACTTCATGGGC |
| | | | TGGTTCAGACAAGCCCCCGGCAAGGAGCTGGAGT |
| | | | GGGTGAGCGTGATCAACTGGAGCGGCGGCACAAG |
| | | | CTACTACAAGGACAGCGTGAAGGGCAGATTCACCA |
| | | | TCAGCAGAGACAACAGCAAGAACACCCTGTACCT |
| | | | GCAGATGAACAGCCTGAGAGCCGAGGACACCGCC |
| | | | GTGTACTACTGCGCCGCCAAGGGCAGCGGCACCTA |
| | | | CGACAGAGAGGGCGAGTACGACTACTGGGGCCAA |
| | | | GGCACCCTGGTGACCGTGAGCAGC |
| BGA-2045 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 48 | VHH AA | EVQLVESGGGLVQPGGSLRLSCAASPFTFSTYFMG |
| | | | WFRQAPGKELEWVSVINWSGGTSYYKDSVKGRF |
| | | | TISRDNSKNTLYLQMNSLRAEDTAVYYCAAKGSG |
| | | | TYDREGEYDYWGQGTLVTVSS |
| | SEQ ID NO: 49 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTTTCACCTTTAGCACCTACTTCATGGGC |
| | | | TGGTTCAGACAAGCCCCCGGCAAGGAGCTGGAGT |
| | | | GGGTGAGCGTGATCAACTGGAGCGGCGGCACAAG |
| | | | CTACTACAAGGACAGCGTGAAGGGCAGATTCACCA |
| | | | TCAGCAGAGACAACAGCAAGAACACCCTGTACCT |
| | | | GCAGATGAACAGCCTGAGAGCCGAGGACACCGCC |
| | | | GTGTACTACTGCGCCGCCAAGGGCAGCGGCACCTA |
| | | | CGACAGAGAGGGCGAGTACGACTACTGGGGCCAA |
| | | | GGCACCCTGGTGACCGTGAGCAGC |
| BGA-2046 | SEQ ID NO: 7 | CDR1 | TYFMG |
| | | (Kabat) | |
| | SEQ ID NO: 8 | CDR2 | VINWSGGTSYYKDSVKG |
| | | (Kabat) | |
| | SEQ ID NO: 9 | CDR3 | KGSGTYDREGEYDY |
| | | (Kabat) | |
| | SEQ ID NO: 50 | VHH AA | EVQLVESGGGLVQPGGSLRLSCAASPRTFQTYFMG |
| | | | WFRQAPGKGREWVSVINWSGGTSYYKDSVKGRF |
| | | | TISRDNSKNTLYLQMNSLRAEDTAVYYCAAKGSG |
| | | | TYDREGEYDYWGQGTLVTVSS |
| | SEQ ID NO: 51 | VHH DNA | GAGGTGCAACTCGTGGAGAGCGGCGGGGGCCTGG |
| | | | TCCAACCCGGGGGCAGCCTGAGACTGAGCTGCGCC |
| | | | GCTAGCCCTAGAACCTTTCAGACCTACTTCATGGGC |
| | | | TGGTTCAGACAAGCCCCCGGCAAGGGCAGAGAGT |
| | | | GGGTGAGCGTGATCAACTGGAGCGGCGGCACAAG |
| | | | CTACTACAAGGACAGCGTGAAGGGCAGATTCACCA |
| | | | TCAGCAGAGACAACAGCAAGAACACCCTGTACCT |
| | | | GCAGATGAACAGCCTGAGAGCCGAGGACACCGCC |
| | | | GTGTACTACTGCGCCGCCAAGGGCAGCGGCACCTA |
| | | | CGACAGAGAGGGCGAGTACGACTACTGGGGCCAA |
| | | | GGCACCCTGGTGACCGTGAGCAGC |

### Example 4. Humanization of BGA-056

For humanization of BGA-056, human germline IgG genes were searched for sequences that share high degrees of homology to the protein sequences of BGA-056 variable regions by blasting against the human immunoglobulin gene database in IMGT. The human IGVH 3-23*01 gene that is present in human antibody repertoires with high frequency and high homology to BGA-056 was selected as the template for humanization.

The humanized variants were constructed in a VHH-human IgG1 Fc format using an in-house developed expression vector, with easy adapting sub-cloning sites. The humanized variants were expressed by transfection of the constructs into Expi293F cells and purified using a protein A column (Cat: 17-5438-02, GE Life Sciences). The purified antibody was concentrated to 0.5-5 mg/ml in PBS and stored in aliquots in a −80° C. freezer.

### Framework Swapping Based on BGA-056

Humanization was carried out by CDR-grafting with key back mutations retained. The humanized VHH antibodies from BGA-056 were engineered in a VHH-human IgG1 Fc format using an in-house developed expression vector. In the initial round of humanization, mutations from camelid variable region to human amino acid residues in framework regions were guided by 3D structure analysis. The camelid framework residues with structural importance for maintaining the canonical structural of CDRs were retained in the first round of humanization, including two amino acid residues—Q94 and R103 (Kabat numbering)—resulting in BGA-174 (Table 10; SEQ ID NOs: 52-53). BGA-174 has comparable binding affinity to the chimeric camelid antibody BGA-056 (Table 10), indicating that this characteristic is retained.

Variants with Point Mutations Based on BGA-174

Single point mutation based on BGA-174 was performed for the above two back mutations to explore their significance (BGA-1741 and BGA-1742; Table 10; SEQ ID NOs: 54 to 57). Binding affinities of BGA-1741 and BGA-1742 were tested by SPR and are shown in Table 10. BGA-174, BGA-1741, and BGA-1742 have comparable affinity to that of BGA-056.

Anti-hu 4Ig-B7H3 VHH antibodies were constructed as human Fc fusion VHH antibody format (VHH-Fc) using in-house developed expression vectors. Expression and preparation of Fc fusion VHH antibodies were achieved by transfection into 293G cells and by purification using a Protein A column (GE Healthcare, 17-5438-02). Humanized BGA-174, BGA-1741, and BGA-1742 exhibit great affinity and high purity, and are easy to purify.

TABLE 10
Binding affinities of variants with point mutations based on BGA-174

| | Mutation | | | | K_{D} ratio to | SEC- |
|---|---|---|---|---|---|---|
| | on BGA- | | | | BGA-056 | HPLC |
| VHH | 056 | kₒₙ (M⁻¹s⁻¹) | k_{off} (s⁻¹) | K_{D} (M) | chimeric | purity |
| BGA-056 | | 1.03E+06 | 1.03E−04 | 1.00E−10 | 1.00 | 88.39 |
| BGA-174 | Q94, | 9.63E+05 | 1.06E−04 | 1.10E−10 | 1.10 | 95.33 |
| | R103 | | | | | |
| BGA-1741 | Q94K | 8.28E+05 | 1.71E−04 | 2.06E−10 | 2.06 | 95.64 |
| BGA-1742 | R103W | 8.40E+05 | 4.00E−04 | 4.76E−10 | 4.76 | 92.64 |

Cell Binding Determination of Humanized Variants from BGA-056

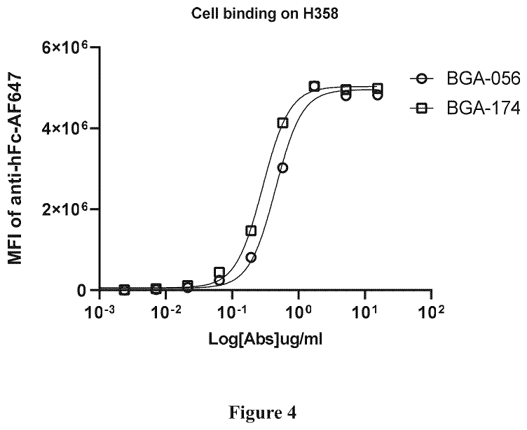

Non-small cell lung cancer cell line H358, which natively expresses high levels of B7H3, was used to evaluate the binding activity of BGA-056 chimeric antibody and humanized variant BGA-174. H358 cells were seeded in a 96-well plate, and were incubated with a diluted series of BGA-056 chimeric antibody or humanized variant BGA-174. Goat anti-human IgG was used as a secondary antibody to detect antibody binding to the cell surface. EC50 values for dose-dependent binding to B7H3 expressing cell lines were determined by fitting the dose-response data to the four-parameter logistic model with GraphPad Prism. The data shown in FIG. 4 and Table 11 demonstrate that the humanized variant BGA-174 exhibits comparable binding affinity to BGA-056 chimeric antibody.

TABLE 11
Binding of chimeric BGA-056 and
humanized VHH BGA-174 to H358

| VHH | H358 binding EC50 (ug/mL) |
|---|---|
| chimeric BGA-056 | 0.452 |
| BGA-174 | 0.289 |

### Affinity Determination of BGA-174 and BGA-174

His-tagged constructs were prepared by adding the 6×His-tag to the C terminus of each of BGA-2042 (SEQ ID NO: 42) (“VHH1_C001”) and BGA-174 (SEQ ID NO: 52) (“VHH1_C002”). Binding affinity and kinetics constants of purified VHH fragments were determined by surface plasmon resonance (Biacore 8K) at 25° C. 4Ig-B7H3 (Cat: 11188-H02H) and 2Ig-B7H3 (Cat: B73-H5253) proteins were coupled onto a CM5 Biocore sensor derivatized via amine coupling with anti-human IgG antibody (Cat: 29234600). The VHH fragments were then flowed through at a rate at 40 μl/min. Association of VHH to B7H3 proteins was monitored for 120 seconds and disassociation of VHH in buffer 1×HBS (0.01 M HEPES, pH 7.4, 0.15 M NaCl, 3 mM EDTA, 0.05% v/v surfactant P20) was monitored for more than 120 seconds.

Ka and Kd were determined by fitting the real-time sensorgram to a 1:1 binding model using Biacore Insight Evaluation software. Binding disassociation equilibrium constants (KD) were calculated from kinetics rates as follows:

$${K}_{D} ( M ) = k d / k a .$$

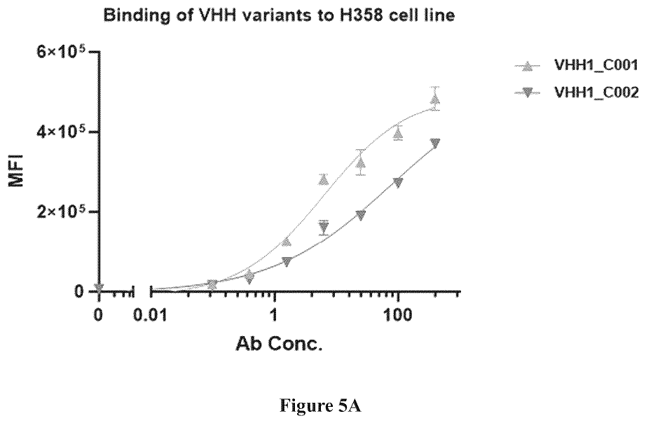

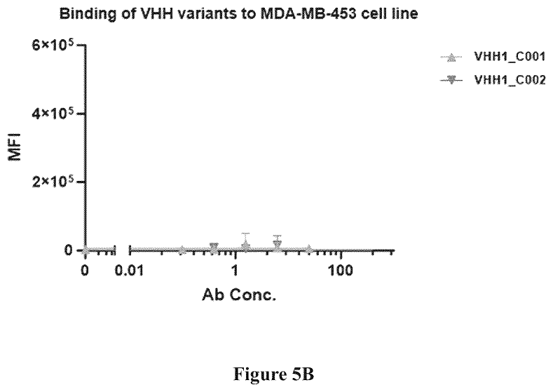

Binding kinetic parameters for the VHH proteins to B7H3 protein are shown in Table 12 and FIGS. 5A-5B.

| Capture 1 | Analyte 1 | | | |
|---|---|---|---|---|
| Solution | Solution | ka (1/Ms) | kd (1/s) | KD (M) |
| B7H3-4IG | VHH1_C001 | 9.14E+05 | 6.77E−03 | 7.41E−09 |
| B7H3-4IG | VHH1_C002 | 2.47E+06 | 9.60E−03 | 3.89E−09 |
| B7H3-2IG | VHH1_C001 | 2.87E+06 | 2.70E−01 | 9.41E−08 |
| B7H3-2IG | VHH1_C002 | 2.99E+06 | 1.29E−01 | 4.33E−08 |

### Binding Activity of VHH Fragments to Native B7H3

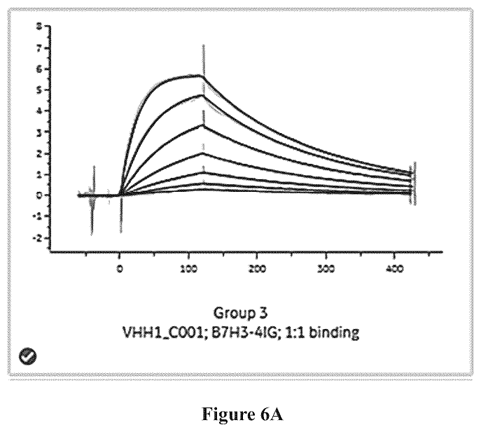

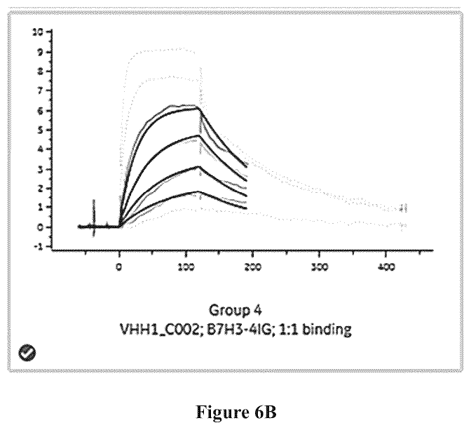

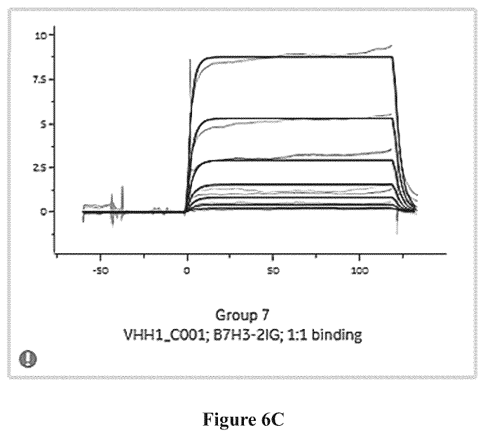

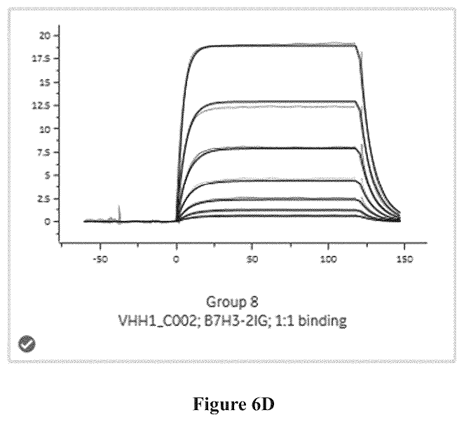

To evaluate the binding ability of anti-B7H3 VHH to B7H3 on the surface of living cells, 1×10⁶ H358 or MDA-MB-453 cells were seeded into 96-well plates. To generate VHH dose response curves, serially diluted VHH proteins (0.2 nM to 200 nM) were added to cells and the bound VHH were detected using His-tag antibody iFluor 488 (Cat No. A01800). Mean fluorescent intensity (MFI) of each cell population was measured using Satorius iQue3 system and EC50 and Emax of each VHH were determined using four parameter logistic model. The results are shown in Table 13 and FIGS. 6A-6D.

TABLE 13
Facs and MFI of dose-dependent binding
of VHH fragments on native B7H3

| Antibody | EC50 (nM) | MFI |
|---|---|---|
| VHH1_C001 | 6.285 | 495973 |
| VHH1_C002 | 77.03 | 539302 |

### Epitope Mapping of the Humanized VHH Fragments

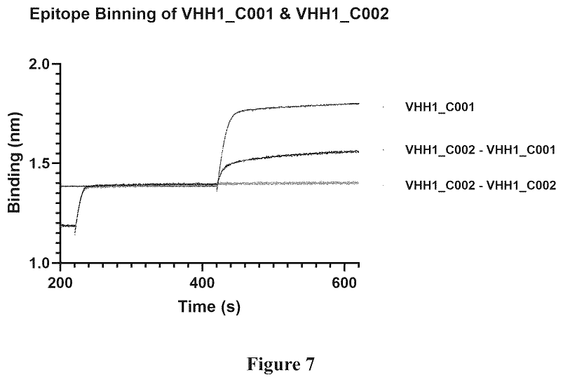

To assess whether the two VHH fragments VHH1_C001 and VHH1_C002 can compete with one another for binding to their respective epitope on B7H3, a binding competition assay was conducted using the tandem method using Octet® RH96. Briefly, the 4Ig-B7H3 proteins (Cat: 11188-H02H) were captured onto Octet® AHC2 Biosensors. The biosensors were then dipped into 100 nM of VHH1_C002 fragments for 180 s. After, the biosensors were dipped into 100 nM of VHH1_C002 and 100 nM VHH1_C001 mixture solution. VHH1_C001 alone or VHH1_C002 alone were tested as control groups. The results are shown in FIG. 7. The results indicate that the epitopes of VHH1_C001 and VHH1_C002 partially compete.

TABLE 14
Amino acid and DNA sequences of the humanized variants of BGA-056

| Antibody | SEQ ID NO | | SEQUENCE |
|---|---|---|---|
| BGA-056 | SEQ ID NO: 12 | CDR1 | DYHMS |
| | | (Kabat) | |
| | SEQ ID NO: 13 | CDR2 | GILNTGLSPTYAATVTG |
| | | (Kabat) | |
| | SEQ ID NO: 14 | CDR3 | GYYRATPSQAN |
| | | (Kabat) | |
| | SEQ ID NO: 15 | VHH AA | EVQLLESGGGLVQPGGSLRLSCAASGFTFSDYHMS |
| | | | WVRQAPGKGLEWVSGILNTGLSPTYAATVTGRFTIS |
| | | | RDNAKNTLYLSMNSLKPEDTAVYYCAQGYYRATPS |
| | | | QANRGQGTQVTVSS |
| | SEQ ID NO: 122 | VHH DNA | GAGGTGCAACTGCTGGAGTCCGGCGGGGGCCTGG |
| | | | TGCAGCCCGGCGGGAGCCTGAGACTGAGCTGCGC |
| | | | CGCTAGCGGCTTCACCTTCAGCGACTACCACATGA |
| | | | GCTGGGTGAGACAAGCCCCCGGCAAGGGCCTGG |
| | | | AGTGGGTGAGCGGCATCCTGAACACCGGCCTGAG |
| | | | CCCCACCTACGCCGCCACCGTGACCGGCAGATTC |
| | | | ACCATCAGCAGAGATAACGCCAAAAACACCCTGT |
| | | | ACCTGAGCATGAACAGCCTGAAGCCCGAGGACAC |
| | | | CGCCGTGTACTACTGCGCCCAAGGCTACTACAGAG |
| | | | CCACCCCTAGCCAAGCCAACAGAGGCCAAGGCAC |
| | | | CCAAGTGACCGTGAGCAGC |
| BGA-174 | SEQ ID NO: 12 | CDR1 | DYHMS |
| | | (Kabat) | |
| | SEQ ID NO: 13 | CDR2 | GILNTGLSPTYAATVTG |
| | | (Kabat) | |
| | SEQ ID NO: 14 | CDR3 | GYYRATPSQAN |
| | | (Kabat) | |
| | SEQ ID NO: 52 | VHH AA | EVQLLESGGGLVQPGGSLRLSCAASGFTFSDYHMS |
| | | | WVRQAPGKGLEWVSGILNTGLSPTYAATVTGRFTIS |
| | | | RDNSKNTLYLQMNSLRAEDTAVYYCAQGYYRATP |
| | | | SQANRGQGTLVTVSS |
| | SEQ ID NO: 53 | VHH DNA | GAAGTGCAGCTGCTGGAATCCGGGGGGGGGCTCG |
| | | | TGCAGCCCGGGGGCAGCCTGAGACTGAGCTGCGC |
| | | | CGCTAGCGGCTTCACCTTCAGCGACTACCACATG |
| | | | AGCTGGGTGAGACAAGCCCCCGGCAAGGGCCTG |
| | | | GAGTGGGTGAGCGGCATCCTGAACACCGGCCTGA |
| | | | GCCCCACCTACGCCGCCACCGTGACCGGCAGATT |
| | | | CACCATCAGCAGAGACAACAGCAAGAACACCCT |
| | | | GTACCTGCAGATGAACAGCCTGAGAGCCGAGGAC |
| | | | ACCGCCGTGTACTACTGCGCCCAAGGCTACTACA |
| | | | GAGCCACCCCTAGCCAAGCCAACAGAGGCCAAG |
| | | | GCACCCTGGTGACCGTGAGCAGC |
| BGA-1741 | SEQ ID NO: 12 | CDR1 | DYHMS |
| | | (Kabat) | |
| | SEQ ID NO: 13 | CDR2 | GILNTGLSPTYAATVTG |
| | | (Kabat) | |
| | SEQ ID NO: 14 | CDR3 | GYYRATPSQAN |
| | | (Kabat) | |
| | SEQ ID NO: 54 | VHH AA | EVQLLESGGGLVQPGGSLRLSCAASGFTFSDYHMS |
| | | | WVRQAPGKGLEWVSGILNTGLSPTYAATVTGRFTIS |
| | | | RDNSKNTLYLQMNSLRAEDTAVYYCAKGYYRATP |
| | | | SQANRGQGTLVTVSS |
| | SEQ ID NO: 55 | VHH DNA | GAAGTGCAGCTGCTGGAATCCGGGGGGGGGCTCG |
| | | | TGCAGCCCGGGGGCAGCCTGAGACTGAGCTGCGC |
| | | | CGCTAGCGGCTTCACCTTCAGCGACTACCACATG |
| | | | AGCTGGGTGAGACAAGCCCCCGGCAAGGGCCTG |
| | | | GAGTGGGTGAGCGGCATCCTGAACACCGGCCTGA |
| | | | GCCCCACCTACGCCGCCACCGTGACCGGCAGATT |
| | | | CACCATCAGCAGAGACAACAGCAAGAACACCCT |
| | | | GTACCTGCAGATGAACAGCCTGAGAGCCGAGGAC |
| | | | ACCGCCGTGTACTACTGCGCCAAGGGCTACTACA |
| | | | GAGCCACCCCTAGCCAAGCCAACAGAGGCCAAG |
| | | | GCACCCTGGTGACCGTGAGCAGC |
| BGA-1742 | SEQ ID NO: 12 | CDR1 | DYHMS |
| | | (Kabat) | |
| | SEQ ID NO: 13 | CDR2 | GILNTGLSPTYAATVTG |
| | | (Kabat) | |
| | SEQ ID NO: 14 | CDR3 | GYYRATPSQAN |
| | | (Kabat) | |
| | SEQ ID NO: 56 | VHH AA | EVQLLESGGGLVQPGGSLRLSCAASGFTFSDYHMS |
| | | | WVRQAPGKGLEWVSGILNTGLSPTYAATVTGRFTIS |
| | | | RDNSKNTLYLQMNSLRAEDTAVYYCAQGYYRATP |
| | | | SQANWGQGTLVTVSS |
| | SEQ ID NO: 57 | VHH DNA | GAAGTGCAGCTGCTGGAATCCGGGGGGGGGCTCG |
| | | | TGCAGCCCGGGGGCAGCCTGAGACTGAGCTGCGC |
| | | | CGCTAGCGGCTTCACCTTCAGCGACTACCACATG |
| | | | AGCTGGGTGAGACAAGCCCCCGGCAAGGGCCTG |
| | | | GAGTGGGTGAGCGGCATCCTGAACACCGGCCTGA |
| | | | GCCCCACCTACGCCGCCACCGTGACCGGCAGATT |
| | | | CACCATCAGCAGAGACAACAGCAAGAACACCCT |
| | | | GTACCTGCAGATGAACAGCCTGAGAGCCGAGGAC |
| | | | ACCGCCGTGTACTACTGCGCCCAAGGCTACTACA |
| | | | GAGCCACCCCTAGCCAAGCCAACTGGGGCCAAGG |
| | | | CACCCTGGTGACCGTGAGCAGC |

1. An antibody or antigen-binding fragment thereof that specifically binds human 4Ig-B7H3, the antibody or antigen-binding fragment thereof comprising:
(1) a heavy chain variable region (VH) that comprises (a) a HCDR1 (Heavy Chain Complementarity Determining Region 1) of SEQ ID NO: 7, (b) a HCDR2 of SEQ ID NO: 8, and (c) a HCDR3 of SEQ ID NO: 9; or
(2) a heavy chain variable region that comprises (a) a HCDR1 of SEQ ID NO: 12, (b) a HCDR2 of SEQ ID NO: 13, and (c) a HCDR3 of SEQ ID NO: 14.

2. The antibody or antigen-binding fragment thereof of claim 1, wherein antibody or antigen-binding fragment thereof comprises:
(1) a heavy chain variable region comprising an amino acid sequence at least 90, 91, 92, 93, 94, 95, 96, 97, 98, or 99% identical to SEQ ID NO: 10;
(2) a heavy chain variable region comprising an amino acid sequence at least 90, 91, 92, 93, 94, 95, 96, 97, 98, or 99% identical to SEQ ID NO: 20;
(3) a heavy chain variable region comprising an amino acid sequence at least 90, 91, 92, 93, 94, 95, 96, 97, 98, or 99% identical to SEQ ID NO: 24;
(4) a heavy chain variable region comprising an amino acid sequence at least 90, 91, 92, 93, 94, 95, 96, 97, 98, or 99% identical to SEQ ID NO: 26;
(5) a heavy chain variable region comprising an amino acid sequence at least 90, 91, 92, 93, 94, 95, 96, 97, 98, or 99% identical to SEQ ID NO: 30;
(6) a heavy chain variable region comprising an amino acid sequence at least 90, 91, 92, 93, 94, 95, 96, 97, 98, or 99% identical to SEQ ID NO: 32;
(7) a heavy chain variable region comprising an amino acid sequence at least 90, 91, 92, 93, 94, 95, 96, 97, 98, or 99% identical to SEQ ID NO: 34;
(8) a heavy chain variable region comprising an amino acid sequence at least 90, 91, 92, 93, 94, 95, 96, 97, 98, or 99% identical to SEQ ID NO: 36;
(9) a heavy chain variable region comprising an amino acid sequence at least 90, 91, 92, 93, 94, 95, 96, 97, 98, or 99% identical to SEQ ID NO: 40;
(10) a heavy chain variable region comprising an amino acid sequence at least 90, 91, 92, 93, 94, 95, 96, 97, 98, or 99% identical to SEQ ID NO: 42;
(11) a heavy chain variable region comprising an amino acid sequence at least 90, 91, 92, 93, 94, 95, 96, 97, 98, or 99% identical to SEQ ID NO: 44;
(12) a heavy chain variable region comprising an amino acid sequence at least 90, 91, 92, 93, 94, 95, 96, 97, 98, or 99% identical to SEQ ID NO: 46;
(13) a heavy chain variable region comprising an amino acid sequence at least 90, 91, 92, 93, 94, 95, 96, 97, 98, or 99% identical to SEQ ID NO: 48;
(14) a heavy chain variable region comprising an amino acid sequence at least 90, 91, 92, 93, 94, 95, 96, 97, 98, or 99% identical to SEQ ID NO: 50;
(15) a heavy chain variable region comprising an amino acid sequence at least 90, 91, 92, 93, 94, 95, 96, 97, 98, or 99% identical to SEQ ID NO: 15;
(16) a heavy chain variable region comprising an amino acid sequence at least 90, 91, 92, 93, 94, 95, 96, 97, 98, or 99% identical to SEQ ID NO: 52;
(17) a heavy chain variable region comprising an amino acid sequence at least 90, 91, 92, 93, 94, 95, 96, 97, 98, or 99% identical to SEQ ID NO: 54; or
(18) a heavy chain variable region comprising an amino acid sequence at least 90, 91, 92, 93, 94, 95, 96, 97, 98, or 99% identical to SEQ ID NO: 56.

3. The antibody or antigen-binding fragment thereof of claim 2, wherein one, two, three, four, five, six, seven, eight, nine, or ten amino acids within at least one of SEQ ID NO: 10, SEQ ID NO: 20, SEQ ID NO: 24, SEQ ID NO: 26, SEQ ID NO: 30, SEQ ID NO: 32, SEQ ID NO: 34, SEQ ID NO: 36, SEQ ID NO: 40, SEQ ID NO: 42, SEQ ID NO: 44, SEQ ID NO: 46, SEQ ID NO: 48, SEQ ID NO: 50, SEQ ID NO: 15, SEQ ID NO: 52, SEQ ID NO: 54, and SEQ ID NO: 56 have been inserted, deleted, or substituted.

4. The antibody or antigen-binding fragment thereof claim 1 of, wherein the antibody or antigen-binding fragment thereof comprises:
(1) a heavy chain variable region comprising SEQ ID NO: 10;
(2) a heavy chain variable region comprising SEQ ID NO: 20;
(3) a heavy chain variable region comprising SEQ ID NO: 24;
(4) a heavy chain variable region comprising SEQ ID NO: 26;
(5) a heavy chain variable region comprising SEQ ID NO: 30;
(6) a heavy chain variable region comprising SEQ ID NO: 32;
(7) a heavy chain variable region comprising SEQ ID NO: 34;
(8) a heavy chain variable region comprising SEQ ID NO: 36;
(9) a heavy chain variable region comprising SEQ ID NO: 40;
(10) a heavy chain variable region comprising SEQ ID NO: 42;
(11) a heavy chain variable region comprising SEQ ID NO: 44;
(12) a heavy chain variable region comprising SEQ ID NO: 46;
(13) a heavy chain variable region comprising SEQ ID NO: 48;
(14) a heavy chain variable region comprising SEQ ID NO: 50;
(15) a heavy chain variable region comprising SEQ ID NO: 15;
(16) a heavy chain variable region comprising SEQ ID NO: 52;
(17) a heavy chain variable region comprising SEQ ID NO: 54; or
(18) a heavy chain variable region comprising SEQ ID NO: 56.

5. The antibody or antigen-binding fragment thereof of claim 1, wherein the antibody or antigen-binding fragment thereof is a monoclonal antibody, a chimeric antibody, a humanized antibody, a human engineered antibody, a single chain antibody (scFv), a Fab fragment, a Fab′ fragment, a F(ab′)2 fragment, a heavy chain antibody (HcAb), or a VHH.

6. The antibody or antigen-binding fragment thereof of claim 5, wherein the antibody or antigen-binding fragment thereof is a VHH.

7. A multi-specific antibody or antigen-binding fragment thereof comprising at least a first antigen binding domain that specifically binds a first human tumor antigen (TAA), wherein the first TAA is human 4Ig-B7H3 and the first antigen binding domain comprises the antibody or antigen-binding fragment thereof of claim 1; and
at least a second antigen binding domain that specifically binds a second human TAA.

8. The multi-specific antibody or antigen-binding fragment thereof of claim 7, wherein the multi-specific antibody is a bispecific antibody.

9. The multi-specific antibody or antigen-binding fragment thereof of claim 7, comprising an amino acid linker, wherein the amino acid linker is any sequence of SEQ ID NO: 79 to SEQ ID NO: 121.

10. The antibody or antigen-binding fragment thereof of claim 1, wherein the antibody or antigen-binding fragment thereof comprises a heavy chain constant region of the subclass of IgG1, IgG2, IgG3, or IgG4, and/or a light chain constant region of the type of kappa or lambda.

11. The antibody or antigen-binding fragment thereof of claim 1, wherein the antibody or antigen-binding fragment thereof comprises a heavy chain constant region of the subclass of IgG1.

12. The antibody or antigen-binding fragment thereof of claim 1, wherein the antibody or antigen-binding fragment thereof has antibody dependent cellular cytotoxicity (ADCC) or complement dependent cytotoxicity (CDC).

13. The antibody or antigen-binding fragment thereof of claim 1, wherein the antibody or antigen-binding fragment thereof has reduced glycosylation or no glycosylation or is hypofucosylated.

14. The antibody or antigen-binding fragment thereof of claim 1, wherein the antibody or antigen-binding fragment thereof comprises increased bisecting GlcNac structures.

15. A pharmaceutical composition comprising the antibody or antigen-binding fragment thereof of claim 1 and a pharmaceutically acceptable carrier.

16. An isolated nucleic acid that encodes the antibody or antigen-binding fragment thereof of claim 1.

17. A vector comprising the nucleic acid of claim 16.

18. A host cell comprising the nucleic acid of claim 16.

19. A process for producing an antibody or antigen-binding fragment thereof comprising cultivating the host cell of claim 18 and recovering the antibody or antibody fragment from the culture.


SEQUENCE LISTING (122 sequences)

SEQ ID NO: 1 - DNA, 1605 bp, Homo sapiens
  source 1..1605: mol_type = genomic DNA; note = Human B7H3-full length
atgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctga

SEQ ID NO: 2 - protein, 534 aa, Homo sapiens
  source 1..534: note = Human B7H3-full length
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 3 - DNA, 702 bp, Homo sapiens
  source 1..702: mol_type = genomic DNA; note = huIgG1 Fc
gctagcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 4 - protein, 234 aa, Homo sapiens
  source 1..234: note = huIgG1 Fc
ASEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 705 bp, Mus sp.
  source 1..705: mol_type = other DNA; note = mIgG2a Fc
gctagcgagcccagagggcccacaatcaagccctgtcctccatgcaaatgcccagcacctaacctcttgggtggaccatccgtcttcatcttccctccaaagatcaaggatgtactcatgatctccctgagccccatggtcacatgtgtggtggtggatgtgagcgaggatgacccagatgtccagatcagctggttcgtgaacaacgtggaagtactcacagctcagacacaaacccatagagaggattacaacagtactctccgggtggtcagtgccctccccatccagcaccaggactggatgagtggcaaggagttcaaatgcaaggtcaacaacaaagccctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgctcagtggtccacgagggtctgcacaatcaccacacgactaagagcttctcccggactccgggtaaa

SEQ ID NO: 6 - protein, 235 aa, Mus sp.
  source 1..235: note = mIgG2a Fc
ASEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 7 - protein, 5 aa, synthetic construct
  source 1..5: note = HCDR1 (Kabat)
TYFMG

SEQ ID NO: 8 - protein, 17 aa, synthetic construct
  source 1..17: note = HCDR2 (Kabat)
VINWSGGTSYYKDSVKG

SEQ ID NO: 9 - protein, 14 aa, synthetic construct
  source 1..14: note = HCDR3 (Chothia); note = HCDR3 (Kabat)
KGSGTYDREGEYDY

SEQ ID NO: 10 - protein, 123 aa, synthetic construct
  source 1..123: note = VHH AA
EVQLVESGGGLVQTGDSLRLSCAASPRTFQTYFMGWFRQAPGKEREFVSVINWSGGTSYYKDSVKGRFTISRDNAKNTVYLQMNNLKPEDTAVYYCAAKGSGTYDREGEYDYWGQGTQVTVSS

SEQ ID NO: 11 - DNA, 369 bp, synthetic construct
  source 1..369: mol_type = other DNA; note = VHH DNA
gaggtgcagctggtggagtctgggggaggattggtgcagactggggactctctgagactctcctgtgcagcctctccacgcaccttccagacctatttcatgggctggttccgccaggctccagggaaggagcgtgagtttgtatcagttattaactggagtggcggtacctcatactataaagattccgtgaagggccgattcaccatctccagagacaacgccaagaataccgtgtatctacaaatgaacaacctgaaacctgaggacacggccgtttattactgtgcagctaaggggtccggtacttacgaccgagagggggaatatgactactggggccaggggacccaggtcaccgtctcctca

SEQ ID NO: 12 - protein, 5 aa, synthetic construct
  source 1..5: note = HCDR1 (Kabat)
DYHMS

SEQ ID NO: 13 - protein, 17 aa, synthetic construct
  source 1..17: note = HCDR2 (Kabat)
GILNTGLSPTYAATVTG

SEQ ID NO: 14 - protein, 11 aa, synthetic construct
  source 1..11: note = HCDR3 (Chothia); note = HCDR3 (Kabat)
GYYRATPSQAN

SEQ ID NO: 15 - protein, 120 aa, synthetic construct
  source 1..120: note = VHH AA
EVQLLESGGGLVQPGGSLRLSCAASGFTFSDYHMSWVRQAPGKGLEWVSGILNTGLSPTYAATVTGRFTISRDNAKNTLYLSMNSLKPEDTAVYYCAQGYYRATPSQANRGQGTQVTVSS

SEQ ID NO: 16 - DNA, 360 bp, synthetic construct
  source 1..360: mol_type = other DNA; note = VHH DNA
gaggtgcagctgctggagtctgggggaggcttggtgcagcctggggggtctctgagactctcctgtgcagcctctggattcaccttcagtgattatcacatgagctgggtccgccaggctccaggaaaggggctcgagtgggtctcaggtattcttaatacaggtcttagtccaacttatgcagccaccgtgacgggccgattcaccatctccagagacaacgccaagaatacgctgtatctctcaatgaacagcctgaaacctgaggacacggccgtgtattattgtgcccagggatactatcgagctaccccctcccaagccaacaggggccaggggacccaggtcaccgtctcctca

SEQ ID NO: 17 - DNA, 696 bp, synthetic construct
  source 1..696: mol_type = other DNA; note = DNA
gaacctaaatcatcagataagactcatacatgccctccttgccctgcacctgaacttcttggaggaccttcagtgttcctatttccaccgaaacctaaagatacacttatgatctcaagaacacctgaagtgacatgcgtggtggtggatgtgtcacacgaagatcctgaagtgaaatttaactggtacgtggatggagtggaagtgcacaacgcaaagaccaagcctagagaagaacaatacaactcaacatacagagtggtgtcagtgcttacagtgcttcaccaagattggcttaacggaaaggagtataaatgcaaagtgtcaaacaaagcacttcctgcacctatcgagaagaccatttcaaaagcaaaaggacaacctagagaacctcaagtgtacacacttcctccttcaagagaagaaatgacaaagaatcaggtgtcgctgacttgtctcgtcaaaggattctatccatcagatatcgcagtggaatgggaatcaaacggacaacctgagaataattacaagactacgcctcctgtgcttgattcagatggatcattcttcctatattcaaagctgacggtggataaatcaagatggcaacaaggaaacgtgttctcctgttcagtgatgcacgaagcacttcacaaccactacacacagaagtccttatcgttgtctcccggaaag

SEQ ID NO: 18 - protein, 232 aa, synthetic construct
  source 1..232: note = AA
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 369 bp, synthetic construct
  source 1..369: mol_type = other DNA; note = VHH DNA
gaagtgcaactggtggagtccggcggcgggctggtccaaaccggggacagcctgagactgagctgcgccgcctcccctagaacctttcagacctacttcatgggctggttcagacaagcccccggcaaggagagagagttcgtgagcgtgatcaactggagcggcggcacaagctactacaaggacagcgtgaagggcagattcaccatcagcagagacaacgccaagaacacagtgtacctgcagatgaacaacctgaagcccgaggacaccgccgtgtactactgcgccgccaagggcagcggcacctacgacagagagggcgagtacgactactggggccaaggcacccaagtgaccgtgagcagc

SEQ ID NO: 20 - protein, 123 aa, synthetic construct
  source 1..123: note = VHH AA
EVQLVESGGGLVQPGGSLRLSCAASPRTFQTYFMGWFRQAPGKEREFVSVINWSGGTSYYKDSVKGRFTISRDNSKNTVYLQMNSLRAEDTAVYYCAAKGSGTYDREGEYDYWGQGTLVTVSS

SEQ ID NO: 21 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttcagacctacttcatgggctggttcagacaagcccccggcaaggagagagagttcgtgagcgtgatcaactggagcggcggcacaagctactacaaggacagcgtgaagggcagattcaccatcagcagagacaacagcaagaacaccgtgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 22 - protein, 123 aa, synthetic construct
  source 1..123: note = VHH AA
EVQLVESGGGLVQPGGSLRLSCAASGRTFQTYFMGWFRQAPGKEREFVSVINWSGGTSYYKDSVKGRFTISRDNSKNTVYLQMNSLRAEDTAVYYCAAKGSGTYDREGEYDYWGQGTLVTVSS

SEQ ID NO: 23 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagcggcagaacctttcagacctacttcatgggctggttcagacaagcccccggcaaggagagagagttcgtgagcgtgatcaactggagcggcggcacaagctactacaaggacagcgtgaagggcagattcaccatcagcagagacaacagcaagaacaccgtgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 24 - protein, 123 aa, synthetic construct
  source 1..123: note = VHH AA
EVQLVESGGGLVQPGGSLRLSCAASPFTFQTYFMGWFRQAPGKEREFVSVINWSGGTSYYKDSVKGRFTISRDNSKNTVYLQMNSLRAEDTAVYYCAAKGSGTYDREGEYDYWGQGTLVTVSS

SEQ ID NO: 25 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctttcacctttcagacctacttcatgggctggttcagacaagcccccggcaaggagagagagttcgtgagcgtgatcaactggagcggcggcacaagctactacaaggacagcgtgaagggcagattcaccatcagcagagacaacagcaagaacaccgtgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 26 - protein, 123 aa, synthetic construct
  source 1..123: note = VHH AA
EVQLVESGGGLVQPGGSLRLSCAASPRTFSTYFMGWFRQAPGKEREFVSVINWSGGTSYYKDSVKGRFTISRDNSKNTVYLQMNSLRAEDTAVYYCAAKGSGTYDREGEYDYWGQGTLVTVSS

SEQ ID NO: 27 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttagcacctacttcatgggctggttcagacaagcccccggcaaggagagagagttcgtgagcgtgatcaactggagcggcggcacaagctactacaaggacagcgtgaagggcagattcaccatcagcagagacaacagcaagaacaccgtgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 28 - protein, 123 aa, synthetic construct
  source 1..123: note = VHH AA
EVQLVESGGGLVQPGGSLRLSCAASPRTFQTYFMGWVRQAPGKEREFVSVINWSGGTSYYKDSVKGRFTISRDNSKNTVYLQMNSLRAEDTAVYYCAAKGSGTYDREGEYDYWGQGTLVTVSS

SEQ ID NO: 29 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttcagacctacttcatgggctgggtgagacaagcccccggcaaggagagagagttcgtgagcgtgatcaactggagcggcggcacaagctactacaaggacagcgtgaagggcagattcaccatcagcagagacaacagcaagaacaccgtgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 30 - protein, 123 aa, synthetic construct
  source 1..123: note = VHH AA
EVQLVESGGGLVQPGGSLRLSCAASPRTFQTYFMGWFRQAPGKGREFVSVINWSGGTSYYKDSVKGRFTISRDNSKNTVYLQMNSLRAEDTAVYYCAAKGSGTYDREGEYDYWGQGTLVTVSS

SEQ ID NO: 31 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttcagacctacttcatgggctggttcagacaagcccccggcaagggcagagagttcgtgagcgtgatcaactggagcggcggcacaagctactacaaggacagcgtgaagggcagattcaccatcagcagagacaacagcaagaacaccgtgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 32 - protein, 123 aa, synthetic construct
  source 1..123: note = VHH AA
EVQLVESGGGLVQPGGSLRLSCAASPRTFQTYFMGWFRQAPGKELEFVSVINWSGGTSYYKDSVKGRFTISRDNSKNTVYLQMNSLRAEDTAVYYCAAKGSGTYDREGEYDYWGQGTLVTVSS

SEQ ID NO: 33 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttcagacctacttcatgggctggttcagacaagcccccggcaaggagctggagttcgtgagcgtgatcaactggagcggcggcacaagctactacaaggacagcgtgaagggcagattcaccatcagcagagacaacagcaagaacaccgtgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 34 - protein, 123 aa, synthetic construct
  source 1..123: note = VHH AA
EVQLVESGGGLVQPGGSLRLSCAASPRTFQTYFMGWFRQAPGKEREWVSVINWSGGTSYYKDSVKGRFTISRDNSKNTVYLQMNSLRAEDTAVYYCAAKGSGTYDREGEYDYWGQGTLVTVSS

SEQ ID NO: 35 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttcagacctacttcatgggctggttcagacaagcccccggcaaggagagagagtgggtgagcgtgatcaactggagcggcggcacaagctactacaaggacagcgtgaagggcagattcaccatcagcagagacaacagcaagaacaccgtgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 36 - protein, 123 aa, synthetic construct
  source 1..123: note = VHH AA
EVQLVESGGGLVQPGGSLRLSCAASPRTFQTYFMGWFRQAPGKEREFVSVINWSGGTSYYKDSVKGRFTISRDNSKNTLYLQMNSLRAEDTAVYYCAAKGSGTYDREGEYDYWGQGTLVTVSS

SEQ ID NO: 37 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttcagacctacttcatgggctggttcagacaagcccccggcaaggagagagagttcgtgagcgtgatcaactggagcggcggcacaagctactacaaggacagcgtgaagggcagattcaccatcagcagagacaacagcaagaacaccctgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 38 - protein, 123 aa, synthetic construct
  source 1..123: note = VHH AA
EVQLVESGGGLVQPGGSLRLSCAASPRTFQTYFMGWFRQAPGKEREFVSVINWSGGTSYYKDSVKGRFTISRDNSKNTVYLQMNSLRAEDTAVYYCAKKGSGTYDREGEYDYWGQGTLVTVSS

SEQ ID NO: 39 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttcagacctacttcatgggctggttcagacaagcccccggcaaggagagagagttcgtgagcgtgatcaactggagcggcggcacaagctactacaaggacagcgtgaagggcagattcaccatcagcagagacaacagcaagaacaccgtgtacctgcagatgaacagcctgagagccgaggacaccgccgtgtactactgcgccaagaagggcagcggcacctacgacagagagggcgagtacgactactggggccaaggcaccctggtgaccgtgagcagc

SEQ ID NO: 40 - protein, 123 aa, synthetic construct
  source 1..123: note = VHH AA
EVQLVESGGGLVQPGGSLRLSCAASPRTFQTYFMGWFRQAPGKGLEWVSVINWSGGTSYYKDSVKGRFTISRDNSKNTLYLQMNSLRAEDTAVYYCAAKGSGTYDREGEYDYWGQGTLVTVSS

SEQ ID NO: 41 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttcagacctacttcatgggctggttcagacaagcccccggcaagggcctggagtgggtgagcgtgatcaactggagcggcggcacaagctactacaaggacagcgtgaagggcagattcaccatcagcagagacaacagcaagaacaccctgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 42 - protein, 123 aa, synthetic construct
  source 1..123: note = VHH AA
EVQLVESGGGLVQPGGSLRLSCAASPRTFQTYFMGWFRQAPGKELEWVSVINWSGGTSYYKDSVKGRFTISRDNSKNTLYLQMNSLRAEDTAVYYCAAKGSGTYDREGEYDYWGQGTLVTVSS

SEQ ID NO: 43 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttcagacctacttcatgggctggttcagacaagcccccggcaaggagctggagtgggtgagcgtgatcaactggagcggcggcacaagctactacaaggacagcgtgaagggcagattcaccatcagcagagacaacagcaagaacaccctgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 44 - protein, 123 aa, synthetic construct
  source 1..123: note = VHH AA
EVQLVESGGGLVQPGGSLRLSCAASPFTFQTYFMGWFRQAPGKELEWVSVINWSGGTSYYKDSVKGRFTISRDNSKNTLYLQMNSLRAEDTAVYYCAAKGSGTYDREGEYDYWGQGTLVTVSS

SEQ ID NO: 45 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctttcacctttcagacctacttcatgggctggttcagacaagcccccggcaaggagctggagtgggtgagcgtgatcaactggagcggcggcacaagctactacaaggacagcgtgaagggcagattcaccatcagcagagacaacagcaagaacaccctgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 46 - protein, 123 aa, synthetic construct
  source 1..123: note = VHH AA
EVQLVESGGGLVQPGGSLRLSCAASPRTFSTYFMGWFRQAPGKELEWVSVINWSGGTSYYKDSVKGRFTISRDNSKNTLYLQMNSLRAEDTAVYYCAAKGSGTYDREGEYDYWGQGTLVTVSS

SEQ ID NO: 47 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttagcacctacttcatgggctggttcagacaagcccccggcaaggagctggagtgggtgagcgtgatcaactggagcggcggcacaagctactacaaggacagcgtgaagggcagattcaccatcagcagagacaacagcaagaacaccctgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 48 - protein, 123 aa, synthetic construct
  source 1..123: note = VHH AA
EVQLVESGGGLVQPGGSLRLSCAASPFTFSTYFMGWFRQAPGKELEWVSVINWSGGTSYYKDSVKGRFTISRDNSKNTLYLQMNSLRAEDTAVYYCAAKGSGTYDREGEYDYWGQGTLVTVSS

SEQ ID NO: 49 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctttcacctttagcacctacttcatgggctggttcagacaagcccccggcaaggagctggagtgggtgagcgtgatcaactggagcggcggcacaagctactacaaggacagcgtgaagggcagattcaccatcagcagagacaacagcaagaacaccctgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 50 - protein, 123 aa, synthetic construct
  source 1..123: note = VHH AA
EVQLVESGGGLVQPGGSLRLSCAASPRTFQTYFMGWFRQAPGKGREWVSVINWSGGTSYYKDSVKGRFTISRDNSKNTLYLQMNSLRAEDTAVYYCAAKGSGTYDREGEYDYWGQGTLVTVSS

SEQ ID NO: 51 - DNA, 369 bp, synthetic construct
  source 1..369: mol_type = other DNA; note = VHH DNA
gaggtgcaactcgtggagagcggcgggggcctggtccaacccgggggcagcctgagactgagctgcgccgctagccctagaacctttcagacctacttcatgggctggttcagacaagcccccggcaagggcagagagtgggtgagcgtgatcaactggagcggcggcacaagctactacaaggacagcgtgaagggcagattcaccatcagcagagacaacagcaagaacaccctgtacctgcagatgaacagcctgagagccgaggacaccgccgtgtactactgcgccgccaagggcagcggcacctacgacagagagggcgagtacgactactggggccaaggcaccctggtgaccgtgagcagc

SEQ ID NO: 52 - protein, 120 aa, synthetic construct
  source 1..120: note = VHH AA
EVQLLESGGGLVQPGGSLRLSCAASGFTFSDYHMSWVRQAPGKGLEWVSGILNTGLSPTYAATVTGRFTISRDNSKNTLYLQMNSLRAEDTAVYYCAQGYYRATPSQANRGQGTLVTVSS

SEQ ID NO: 53 - DNA, 360 bp, synthetic construct
  source 1..360: mol_type = other DNA; note = VHH DNA
gaagtgcagctgctggaatccgggggggggctcgtgcagcccgggggcagcctgagactgagctgcgccgctagcggcttcaccttcagcgactaccacatgagctgggtgagacaagcccccggcaagggcctggagtgggtgagcggcatcctgaacaccggcctgagccccacctacgccgccaccgtgaccggcagattcaccatcagcagagacaacagcaagaacaccctgtacctgcagatgaacagcctgagagccgaggacaccgccgtgtactactgcgcccaaggctactacagagccacccctagccaagccaacagaggccaaggcaccctggtgaccgtgagcagc

SEQ ID NO: 54 - protein, 120 aa, synthetic construct
  source 1..120: note = VHH AA
EVQLLESGGGLVQPGGSLRLSCAASGFTFSDYHMSWVRQAPGKGLEWVSGILNTGLSPTYAATVTGRFTISRDNSKNTLYLQMNSLRAEDTAVYYCAKGYYRATPSQANRGQGTLVTVSS

SEQ ID NO: 55 - DNA, 360 bp, synthetic construct
  source 1..360: mol_type = other DNA; note = VHH DNA
gaagtgcagctgctggaatccgggggggggctcgtgcagcccgggggcagcctgagactgagctgcgccgctagcggcttcaccttcagcgactaccacatgagctgggtgagacaagcccccggcaagggcctggagtgggtgagcggcatcctgaacaccggcctgagccccacctacgccgccaccgtgaccggcagattcaccatcagcagagacaacagcaagaacaccctgtacctgcagatgaacagcctgagagccgaggacaccgccgtgtactactgcgccaagggctactacagagccacccctagccaagccaacagaggccaaggcaccctggtgaccgtgagcagc

SEQ ID NO: 56 - protein, 120 aa, synthetic construct
  source 1..120: note = VHH AA
EVQLLESGGGLVQPGGSLRLSCAASGFTFSDYHMSWVRQAPGKGLEWVSGILNTGLSPTYAATVTGRFTISRDNSKNTLYLQMNSLRAEDTAVYYCAQGYYRATPSQANWGQGTLVTVSS

SEQ ID NO: 57 - DNA, 360 bp, synthetic construct
  source 1..360: mol_type = other DNA; note = VHH DNA
gaagtgcagctgctggaatccgggggggggctcgtgcagcccgggggcagcctgagactgagctgcgccgctagcggcttcaccttcagcgactaccacatgagctgggtgagacaagcccccggcaagggcctggagtgggtgagcggcatcctgaacaccggcctgagccccacctacgccgccaccgtgaccggcagattcaccatcagcagagacaacagcaagaacaccctgtacctgcagatgaacagcctgagagccgaggacaccgccgtgtactactgcgcccaaggctactacagagccacccctagccaagccaactggggccaaggcaccctggtgaccgtgagcagc

SEQ ID NO: 58 - protein, 438 aa, synthetic construct
  source 1..438: note = AA
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEA

SEQ ID NO: 59 - protein, 8 aa, synthetic construct
  source 1..8: note = HCDR1 IMGT
PRTFQTYF

SEQ ID NO: 60 - protein, 8 aa, synthetic construct
  source 1..8: note = HCDR2 IMGT
INWSGGTS

SEQ ID NO: 61 - protein, 16 aa, synthetic construct
  source 1..16: note = HCDR3 IMGT
AAKGSGTYDREGEYDY

SEQ ID NO: 62 - protein, 7 aa, synthetic construct
  source 1..7: note = HCDR1 Chothia
PRTFQTY

SEQ ID NO: 63 - protein, 6 aa, synthetic construct
  source 1..6: note = HCDR2 (Chothia)
NWSGGT

SEQ ID NO: 64 - protein, 8 aa, synthetic construct
  source 1..8: note = HCDR1 IMGT
GRTFQTYF

SEQ ID NO: 65 - protein, 7 aa, synthetic construct
  source 1..7: note = HCDR1 (Chothia)
GRTFQTY

SEQ ID NO: 66 - protein, 8 aa, synthetic construct
  source 1..8: note = HCDR1 (IMGT)q
PFTFQTYF

SEQ ID NO: 67 - protein, 7 aa, synthetic construct
  source 1..7: note = HCDR1 (Chothia)
PFTFQTY

SEQ ID NO: 68 - protein, 8 aa, synthetic construct
  source 1..8: note = HCDR1 (IMGT)
PRTFSTYF

SEQ ID NO: 69 - protein, 7 aa, synthetic construct
  source 1..7: note = HCDR1 (Chothia)
PRTFSTY

SEQ ID NO: 70 - protein, 16 aa, synthetic construct
  source 1..16: note = HCDR3 (IMGT)
AKKGSGTYDREGEYDY

SEQ ID NO: 71 - protein, 8 aa, synthetic construct
  source 1..8: note = HCDR1 (IMGT)
PFTFSTYF

SEQ ID NO: 72 - protein, 7 aa, synthetic construct
  source 1..7: note = PFTFSTY
PFTFSTY

SEQ ID NO: 73 - protein, 8 aa, synthetic construct
  source 1..8: note = HCDR1 (IMGT)
GFTFSDYH

SEQ ID NO: 74 - protein, 8 aa, synthetic construct
  source 1..8: note = HCDR2 (IMGT)
ILNTGLSP

SEQ ID NO: 75 - protein, 13 aa, synthetic construct
  source 1..13: note = HCDR3 (IMGT)
AQGYYRATPSQAN

SEQ ID NO: 76 - protein, 7 aa, synthetic construct
  source 1..7: note = HCDR1 (Chothia)
GFTFSDY

SEQ ID NO: 77 - protein, 6 aa, synthetic construct
  source 1..6: note = HCDR2 (Chothia)
LNTGLS

SEQ ID NO: 78 - protein, 13 aa, synthetic construct
  source 1..13: note = HCDR3 (IMGT)
AKGYYRATPSQAN

SEQ ID NO: 79-83 - skipped (5 entries)

SEQ ID NO: 84 - protein, 4 aa, synthetic construct
  source 1..4: note = linker
GGGS

SEQ ID NO: 85 - protein, 4 aa, synthetic construct
  source 1..4: note = Linker
SGGG

SEQ ID NO: 86 - protein, 5 aa, synthetic construct
  source 1..5: note = linker
GGGGS

SEQ ID NO: 87 - protein, 7 aa, synthetic construct
  source 1..7: note = linker
GGGGSGS

SEQ ID NO: 88 - protein, 7 aa, synthetic construct
  source 1..7: note = linker
GGGGSGS

SEQ ID NO: 89 - protein, 8 aa, synthetic construct
  source 1..8: note = linker
GGGGSGGS

SEQ ID NO: 90 - protein, 10 aa, synthetic construct
  source 1..10: note = linker
GGGGSGGGGS

SEQ ID NO: 91 - protein, 15 aa, synthetic construct
  source 1..15: note = linker
GGGGSGGGGSGGGGS

SEQ ID NO: 92 - protein, 16 aa, synthetic construct
  source 1..16: note = linker
AKTTPKLEEGEFSEAR

SEQ ID NO: 93 - protein, 17 aa, synthetic construct
  source 1..17: note = linker
AKTTPKLEEGEFSEARV

SEQ ID NO: 94 - protein, 9 aa, synthetic construct
  source 1..9: note = linker
AKTTPKLGG

SEQ ID NO: 95 - protein, 10 aa, synthetic construct
  source 1..10: note = linker
SAKTTPKLGG

SEQ ID NO: 96 - protein, 17 aa, synthetic construct
  source 1..17: note = linker
AKTTPKLEEGEFSEARV

SEQ ID NO: 97 - protein, 6 aa, synthetic construct
  source 1..6: note = linker
SAKTTP

SEQ ID NO: 98 - protein, 10 aa, synthetic construct
  source 1..10: note = SAKTTPKLGG
SAKTTPKLGG

SEQ ID NO: 99 - protein, 6 aa, synthetic construct
  source 1..6: note = RADAAP
RADAAP

SEQ ID NO: 100 - protein, 9 aa, synthetic construct
  source 1..9: note = RADAAPTVS
RADAAPTVS

SEQ ID NO: 101 - protein, 12 aa, synthetic construct
  source 1..12: note = RADAAAAGGPGS
RADAAAAGGPGS

SEQ ID NO: 102 - protein, 27 aa, synthetic construct
  source 1..27: note = Linker
RADAAAAGGGGSGGGGSGGGGSGGGGS

SEQ ID NO: 103 - protein, 6 aa, synthetic construct
  source 1..6: note = SAKTTP
SAKTTP

SEQ ID NO: 104 - protein, 10 aa, synthetic construct
  source 1..10: note = linker
SAKTTPKLGG

SEQ ID NO: 105 - protein, 18 aa, synthetic construct
  source 1..18: note = linker
SAKTTPKLEEGEFSEARV

SEQ ID NO: 106 - protein, 5 aa, synthetic construct
  source 1..5: note = linker
ADAAP

SEQ ID NO: 107 - protein, 12 aa, synthetic construct
  source 1..12: note = linker
ADAAPTVSIFPP

SEQ ID NO: 108 - protein, 5 aa, synthetic construct
  source 1..5: note = linker
TVAAP

SEQ ID NO: 109 - protein, 12 aa, synthetic construct
  source 1..12: note = TVAAPSVFIFPP
TVAAPSVFIFPP

SEQ ID NO: 110 - protein, 6 aa, synthetic construct
  source 1..6: note = QPKAAP
QPKAAP

SEQ ID NO: 111 - protein, 13 aa, synthetic construct
  source 1..13: note = QPKAAPSVTLFPP
QPKAAPSVTLFPP

SEQ ID NO: 112 - protein, 6 aa, synthetic construct
  source 1..6: note = linker
AKTTPP

SEQ ID NO: 113 - protein, 13 aa, synthetic construct
  source 1..13: note = AKTTPPSVTPLAP
AKTTPPSVTPLAP

SEQ ID NO: 114 - protein, 6 aa, synthetic construct
  source 1..6: note = linker
AKTTAP

SEQ ID NO: 115 - protein, 13 aa, synthetic construct
  source 1..13: note = linker
AKTTAPSVYPLAP

SEQ ID NO: 116 - protein, 6 aa, synthetic construct
  source 1..6: note = ASTKGP
ASTKGP

SEQ ID NO: 117 - protein, 13 aa, synthetic construct
  source 1..13: note = linker
ASTKGPSVFPLAP

SEQ ID NO: 118 - protein, 15 aa, synthetic construct
  source 1..15: note = GENKVEYAPALMALS
GENKVEYAPALMALS

SEQ ID NO: 119 - protein, 15 aa, synthetic construct
  source 1..15: note = linker
GPAKELTPLKEAKVS

SEQ ID NO: 120 - protein, 15 aa, synthetic construct
  source 1..15: note = linker
GHEAAAVMQVQYPAS

SEQ ID NO: 121 - protein, 20 aa, synthetic construct
  source 1..20: note = linker
GGGGSGGGGSGGGGSGGGGS

SEQ ID NO: 122 - DNA, 360 bp, synthetic construct
  source 1..360: mol_type = other DNA; note = VHH DNA
gaggtgcaactgctggagtccggcgggggcctggtgcagcccggcgggagcctgagactgagctgcgccgctagcggcttcaccttcagcgactaccacatgagctgggtgagacaagcccccggcaagggcctggagtgggtgagcggcatcctgaacaccggcctgagccccacctacgccgccaccgtgaccggcagattcaccatcagcagagataacgccaaaaacaccctgtacctgagcatgaacagcctgaagcccgaggacaccgccgtgtactactgcgcccaaggctactacagagccacccctagccaagccaacagaggccaaggcacccaagtgaccgtgagcagc